(19) United States
(12) Patent Application Publication
(10) Pub. No.: US 2024/0261238 A1
(43) Pub. Date: Aug. 8, 2024

(54) METHOD OF TREATING DRUG RESISTANT EPILEPSY

(71) Applicant: ICAHN SCHOOL OF MEDICINE AT MOUNT SINAI, New York, NY (US)

(72) Inventors: Madeline C. Fields, Tenafly, NJ (US); Lara Marcuse, New York, NY (US)

(21) Appl. No.: 18/564,527

(22) PCT Filed: May 27, 2022
(86) PCT No.: PCT/US2022/031245

§ 371 Date: Nov. 27, 2023

Related U.S. Application Data
(60) Provisional application No. 63/194,586, filed on May 28, 2021.

(51) Int. Cl. A61K 31/135; A61K 45/06

(52) U.S. Cl. CPC A61K 31/135; A61K 45/06

(57) ABSTRACT

Disclosed herein is a method of treating Drug Resistant Epilepsy with ketamine.

### CROSS-REFERENCE TO RELATED APPLICATIONS

This application claims the benefit of U.S. Provisional Application No. 63/194,586, filed May 28, 2021, the entire disclosure of which is incorporated herein by reference.

### SUMMARY

Disclosed herein is a method of treating drug resistant epilepsy in a subject in need of treatment thereof, comprising administering a therapeutically effective amount of ketamine, or a pharmaceutically acceptable salt thereof, to the subject.

Disclosed herein is a method of treating epilepsy in a subject in need of treatment thereof, comprising administering a therapeutically effective amount of ketamine, or a pharmaceutically acceptable salt thereof, to the subject; wherein the subject experiences substantially no anesthesia after administration of the ketamine or pharmaceutically acceptable salt thereof.

In some embodiments, the subject was previously administered an anti-seizure drug.

Disclosed herein is a method of treating epilepsy in a subject in need of treatment thereof, comprising administering a therapeutically effective amount of ketamine, or a pharmaceutically acceptable salt thereof, to the subject; wherein the subject was previously administered an anti-seizure drug.

In some embodiments, substantially no improvement in the duration, severity, and/or frequency of seizures was observed in the subject after administration of the anti-seizure drug.

Disclosed herein is a method of treating epilepsy in a subject in need of treatment thereof, comprising administering a therapeutically effective amount of ketamine, or a pharmaceutically acceptable salt thereof, to the subject; wherein the subject has a clinical record that indicates that the subject has epilepsy and was administered an anti-seizure drug. In some embodiments, the clinical record indicates that substantially no improvement in the duration, severity, and/or frequency of seizures was observed in the subject after administration of the anti-seizure drug.

Disclosed herein is a method of treating epilepsy in a subject in need of treatment thereof, comprising
determining that the subject was previously administered an anti-seizure drug;
determining that there was substantially no improvement in the duration, severity, and/or frequency of seizures in the subject after administration of the anti-seizure drug; and
administering a therapeutically effective amount of ketamine, or a pharmaceutically acceptable salt thereof, to the subject.

Disclosed herein is a method of selecting a subject for treatment including administration of a therapeutically effective amount of ketamine, or a pharmaceutically acceptable salt thereof, the method comprising:
identifying a subject that has epilepsy and was administered an anti-seizure drug;
determining that there was substantially no improvement in the duration, severity, and/or frequency of seizures in the subject after administration of the anti-seizure drug; and
selecting the subject for treatment including administration of a therapeutically effective amount of ketamine, or a pharmaceutically acceptable salt thereof.

In some embodiments, the anti-seizure drug is selected from the group consisting of topiramate, valproic acid, felbamate, rufinamide, stiripentol, fenfluramine, clobazam, clonazepam, lorazepam, gabapentin, pregabalin, retigabine, phenytoin, carbamazepine, oxcarbazepine, eslicarbazepine acetate, lamotrigine, lacosamide, zonisamide, levetiracetam, brivaracetam, ethosuximide, perampanel, phenobarbital, primidone, epidiolex, cenobamate, and tiagabine.

In some embodiments, after administering the ketamine the frequency, severity, and/or duration of one or more symptoms of the epilepsy is reduced. In some embodiments, the one or more symptoms of the epilepsy are selected from the group consisting of: focal seizures, generalized seizures, non-convulsive seizures, involuntary muscle contractions, auras, Jacksonian march, sensory disturbances, lip smacking, object lifting, involuntary vocalizations, erratic breathing, blue skin, loss of bladder or bowel control, tongue biting, or any combination thereof. In some embodiments, the focal seizure is a seizure of the left hemisphere of the brain. In some embodiments, the focal seizure is a seizure of the right hemisphere of the brain.

In some embodiments, the generalized seizures are selected from the group consisting of tonic-clonic, tonic, clonic, myoclonic, absence, and atonic seizures.

In some embodiments, the subject experiences substantially no anesthesia after administration of the ketamine.

In some embodiments, the therapeutically effective amount is a sub-anesthetic dose of ketamine. In some embodiments, the therapeutically effective amount is from about 10% to about 30% of the dose required to produce anesthesia in a subject.

In some embodiments, the subject receives multiple doses of ketamine at spaced apart intervals.

In some embodiments, the ketamine is administered intravenously. In some embodiments, the ketamine is formulated with a pharmaceutically acceptable carrier or diluent. In some embodiments, the carrier or diluent is aqueous. In some embodiments, the carrier or diluent comprises sterile phosphate buffered saline solution, bacteriostatic water, aqueous glycine, or any combination thereof. In some embodiments, the therapeutically effective amount is between about 0.1 and about 2.0 mg/kg. In some embodiments, the therapeutically effective amount is about 0.5 ml/kg.

In some embodiments, the ketamine is administered intranasally. In some embodiments, the ketamine is formulated as a solution or suspension. In some embodiments, the ketamine is administered as an aerosol spray. In some embodiments, the ketamine is formulated as a dry powder. In some embodiments, the ketamine is contacted with the nasal mucosa.

In some embodiments, the ketamine is administered by means of a device comprising a metered dose inhaler. In some embodiments, the ketamine or pharmaceutically acceptable salt thereof is administered by means of a device comprising a nasal spray inhaler containing an aerosol spray formulation of ketamine and a pharmaceutically acceptable dispersant, wherein the device is metered to disperse an amount of the aerosol formulation by forming a spray that contains a dose of ketamine effective to treat epilepsy but which dose of ketamine is determined by a physician or medical care provider to be below a dose that causes anesthesia.

In some embodiments, the ketamine is formulated with a pharmaceutically acceptable carrier or diluent. In some embodiments, the ketamine is formulated with a dispersant. In some embodiments, the ketamine is formulated with a mucosal penetration enhancer. In some embodiments, the ketamine is formulated with a propellant.

In some embodiments, the therapeutically effective amount is between about 0.05 and about 0.7 mg/kg. In some embodiments, the therapeutically effective amount is about 0.5 ml/kg.

In some embodiments, the ketamine is administered 3 times per week. In some embodiments, the ketamine is administered 3 times per week for about 1 month then 2 times per week for about 1 month.

In some embodiments, the ketamine is administered during a seizure, and the subject transitions to a postictal state or a normal state within about 10 minutes after administration of the ketamine. In some embodiments, the ketamine is administered during a seizure, and the subject transitions to a postictal state or a normal state within 5 minutes after administration of the ketamine.

In some embodiments, the ketamine is esketamine

In some embodiments, after administering the ketamine the frequency of seizures is reduced. In some embodiments, the frequency of seizures is assessed by a seizure diary. In some embodiments, the reduction in seizure frequency is at least about 10%. In some embodiments, the reduction in seizure frequency is at least about 25%. In some embodiments, the reduction in seizure frequency is at least about 50%.

In some embodiments, the subject was identified or diagnosed as having depression and/or anxiety before administering the ketamine.

In some embodiments, the Neurological Disorders Depression Inventory for Epilepsy (NDDI-E) score of the subject is lower after administering the ketamine. In some embodiments, the NDDI-E score is at least 1 point lower after administering the ketamine. In some embodiments, the NDDI-E score is at least 3 points lower after administering the ketamine.

In some embodiments, the Generalized Anxiety Disorder 7 (GAD-7) score of the subject is lower after administering the ketamine. In some embodiments, the GAD-7 score is at least 10% lower after administering the ketamine. In some embodiments, the GAD-7 score is at least 30% lower after administering the ketamine.

In some embodiments, the Anxiety, Depression and Mood Scale (ADAMS) score of the subject is lower after administering the ketamine. In some embodiments, the ADAMS score is at least 10% lower after administering the ketamine. In some embodiments, the ADAMS score is at least 30% lower after administering the ketamine.

In some embodiments, the Quality of Life in Epilepsy Inventory-10 (QOLIE-10) score of the subject is lower after administering the ketamine. In some embodiments, the QOLIE-10 score is at 10% lower after administering the ketamine. In some embodiments, the QOLIE-10 score is at least 30% lower after administering the ketamine.

In some embodiments, the second therapeutic agent is an anti-seizure drug.

### Definitions

As used herein, the terms “about” and “approximately” are used interchangeably, and when used to refer to modify a numerical value, encompass a range of uncertainty of the numerical value of from 0% to 10% of the numerical value.

For purposes of clarification, “the subject experiences substantially no anesthesia” is understood to mean that the subject exhibits behaviors indicative of minimal to no anesthesia. For example, the subject responds to verbal questions or requests and/or painful stimuli that, in the judgment of a physician or medical care provider, indicates minimal to no anesthesia. In some embodiments, a neurological examination of the mental status of the subject provides a characterization of “alert” or “lethargic”. In some embodiments, a neurological examination of the mental status of the subject provides a characterization of other than “obtunded”, “stupor”, or “coma”.

As used herein, “substantially no improvement in the duration, severity, and/or frequency of seizures” refers to no improvement, minimal improvement (e.g., less than about 10% improvement, less than about 8% improvement, less than about 5% improvement, less than about 2% improvement, or about 0% improvement) or a deterioration in one or more of the duration, severity, and frequency of seizures relative to baseline (i.e., the duration, severity, and frequency of seizures assessed before administration of the anti-seizure drug). Improvement in the duration, severity, and/or frequency of seizures is assessed by means of a seizure diary. A seizure diary is a document (e.g., a notebook and/or calendar) or application for, e.g., the subject, a physician, and/or a medical care provider to note frequency and severity of seizures. The frequency and severity of seizures can, e.g., be reported immediately after their occurrence, or on a regular basis. Exemplary seizure diaries include, for example, Epsy (https://www.epsyhealth.com/) or Seizure Tracker (https://seizuretracker.com/).

As used herein, “postictal state” refers to a state of consciousness that occurs after an epileptic seizure during which the subject is recovering from the seizure. During a postictal state, the subject experiences, for example, drowsiness, confusion, nausea, hypertension, headache or migraine, and disorientation. In some embodiments, the duration of the postictal state is from about 3 minutes to about 3 hours (e.g., from about 3 minutes to about 10 minutes, from about 10 minutes to about 20 minutes, from about 20 minutes to about 40 minutes, from about 40 minutes to about 1 hour, from about 1 hour to about 2 hours, or from about 2 hours to about 3 hours). In some embodiments, the subject's brain activity and/or behaviors return to normal (e.g., what is routinely observed in the subject when the subject is awake and not experiencing a seizure, a post-ictal state, or a pre-ictal state) after the postictal state.

As used herein, a “therapeutically effective amount” of a drug is an amount effective to demonstrate a desired activity of the drug. In some embodiments, a therapeutically effective amount of ketamine is an amount effective to alleviate, i.e., observably reduce, the symptoms of drug resistant epilepsy.

As used herein, the term “aerosol” refers to suspension in the air. In particular, aerosol refers to the particlization or atomization of a formulation of the invention and its suspension in the air. According to the present invention, an aerosol formulation is a formulation comprising ketamine for nasal inhalation or pulmonary administration.

As used herein, the term “inhaler” refers both to devices for nasal and pulmonary administration of a drug, e.g., in solution, powder and the like. For example, a the term “inhaler” is intended to encompass a propellant driven inhaler, such as is used for to administer antihistamine for acute asthma attacks, and plastic spray bottles, such as are used to administer decongestants.

As used herein, the term “dispersant” refers to an agent that assists aerosolization of the ketamine or absorption of the ketamine in mucosal tissue, or both. In a specific aspect, the dispersant can be a mucosal penetration enhancer. Preferably, the dispersant is pharmaceutically acceptable.

As used herein, the term “pharmaceutically acceptable” means approved by a regulatory agency of the Federal or a state government or listed in the U.S. Pharmacopeia or other generally recognized pharmacopeia for use in animals (e.g., humans).

As used herein, “ketamine” includes preparations of ketamine that contain a racemic mixture of S(+) and R(−) stereoisomers of ketamine, preparations that contain differences in the enantiomeric proportions of the S(+) and R(−) stereoisomers, and preparations that contain only one of the enantiomers (e.g., only S(+) ketamine or only R(−) ketamine).

### DESCRIPTION OF DRAWINGS

FIG. 1 is a flowchart depicting activities at each patient visit in a study assessing the efficacy of sub-anesthetic doses of ketamine in treating drug-resistant epilepsy.

### DETAILED DESCRIPTION

Epilepsy is a neurological disorder in which abnormal brain activity leads to seizures. While some patients can be treated successfully for epileptic seizures, others have seizures that do not respond to treatment, i.e. are epileptic seizures resistant to treatment with anti-seizure drugs.

Glutamate is the predominant excitatory neurotransmitter in the adult brain. In both animal and human models glutamate is involved in seizures and involved in the process that causes recurrent seizures (epileptogenesis). Glutamate is also likely involved in several comorbid conditions that affect the epilepsy population including depression (Niciu M J et al, 2015; Zarate C A et al, 2006).

Ketamine is a noncompetitive NMDA receptor antagonist that blocks NMDA receptor-mediated glutamatergic neurotransmission (Dingledine et al, 1999; Freeman F G et al, 1982, Aram J A et al, 1989). Ketamine may be neuroprotective by blocking glutamate mediated NMDA receptor-induced neurotoxicity (Mazarati A M et al, 1999; Kapur J et al, 1990, Fujikawa D G, 1995). There are several case reports, 3 retrospective studies and 2 prospective studies examining anesthetic doses of ketamine in refractory and super refractory status epilepticus (Gaspard N et al, 2013; Rosati A et al, 2018; Mewasingh L D et al, 2003; Tarocco A et al, 2014; Synowiec A S et al, 2013, Zieler F A et al, 2013, Esaian D et al, 2013; Kramer A H, 2012; Yeh P S et al, 2011; Hsieh C Y et al, 2010; Pruss H et al, 2008; Kramer U et al, 2005; Ubogu E E et al, 2003; Sheht R D et al, 1998; Walker M C et al, 1996, Zieler F A et al, 2014). However, ketamine has never been used as a conventional anti-seizure medication (ASM) at subanesthetic doses in patients with drug-resistant epilepsy (DRE). DRE composes >30% of the epilepsy population. Despite the introduction of multiple new ASMs, many with novel mechanisms of action, the percentage of people with DRE has not diminished (Brodie M J, 2013). Patients with DRE are at increased risk of premature death (sudden unexpected or unexplained death in epilepsy patients SUDEP) (Mohanraj R et al, 2006), injuries, psychosocial dysfunction and decreased quality of life (Lawn N D et al 2004, McCagh J et al, 2009).

Treatment resistant depression and DRE have a number of things in common including possible mechanism of action—glutamate. A body of research has shown that subanesthetic doses of ketamine can successfully treat drug resistant depression (Niciu et al, 2013; Lent J K et al, 2019; Lapidus K A et al, 2014; Murrough J W et al, 2012).

People with epilepsy have much higher rates of anxiety and depression than the general population, with a recent meta-analysis finding that 20.2% of people with epilepsy suffer from anxiety and 22.9% suffer from depression (Scott A J et al, 2017). Prior to this decade, it was thought that the burden of epilepsy was the cause of the increase in affective disorders. However, recent investigations have elucidated a more complex and bi-directional relationship. Specifically, people with epilepsy have a higher incidence of psychosis, depression, anxiety, and suicidal ideation both before and after the diagnosis of epilepsy (Hesdorffer D C et al, 2012). This suggests that psychiatric illness and epilepsy may share common underlying neurobiology. Furthermore, psychiatric illness may in ways that are not yet understood lower the seizure threshold. Multiple medications used for mood stabilization are also ASMs. As people with epilepsy do suffer from high rates of anxiety and depression, the secondary aim is to investigate if treatment with ketamine has any benefit on these patients' mood and quality of life.

Ketamine is a safe, inexpensive, readily available drug, with minor to moderate adverse side effects. The present invention is based on the surprising and unexpected discovery that administration of ketamine can alleviate symptoms of epilepsy (e.g., seizures) in subjects that are refractory to other anti-seizure drugs.

Herein is disclosed a method of treating drug resistant epilepsy in a subject in need of treatment thereof, comprising administering a therapeutically effective amount of ketamine, or a pharmaceutically acceptable salt thereof, to the subject.

Herein is disclosed a method of treating epilepsy in a subject in need of treatment thereof, comprising administering a therapeutically effective amount of ketamine, or a pharmaceutically acceptable salt thereof, to the subject; wherein the subject experiences substantially no anesthesia after administration of the ketamine or pharmaceutically acceptable salt thereof.

Herein is disclosed a method of treating one or more symptoms of epilepsy in a subject in need of treatment thereof, comprising administering a therapeutically effective amount of ketamine, or a pharmaceutically acceptable salt thereof, to the subject.

In some embodiments, the subject was previously administered an anti-seizure drug.

Herein is disclosed a method of treating epilepsy in a subject in need of treatment thereof, comprising administering a therapeutically effective amount of ketamine, or a pharmaceutically acceptable salt thereof, to the subject; wherein the subject was previously administered an anti-seizure drug.

In some embodiments, substantially no improvement in the duration, severity, and/or frequency of seizures was observed in the subject after administration of the anti-seizure drug.

Herein is disclosed a method of treating epilepsy in a subject in need of treatment thereof, comprising administering a therapeutically effective amount of ketamine, or a pharmaceutically acceptable salt thereof, to the subject; wherein the subject has a clinical record that indicates that the subject has epilepsy and was administered an anti-seizure drug.

In some embodiments, the clinical record indicates that substantially no improvement in the duration, severity, and/or frequency of seizures was observed in the subject after administration of the anti-seizure drug.

Herein is disclosed a method of treating epilepsy in a subject in need of treatment thereof, comprising
determining that the subject was previously administered an anti-seizure drug;
determining that there was substantially no improvement in the duration, severity, and/or frequency of seizures in the subject after administration of the anti-seizure drug; and
administering a therapeutically effective amount of ketamine, or a pharmaceutically acceptable salt thereof, to the subject.

Herein is disclosed a method of selecting a subject for treatment including administration of a therapeutically effective amount of ketamine, or a pharmaceutically acceptable salt thereof, the method comprising:
identifying a subject that has epilepsy and was administered an anti-seizure drug;
determining that there was substantially no improvement in the duration, severity, and/or frequency of seizures in the subject after administration of the anti-seizure drug; and
selecting the subject for treatment including administration of a therapeutically effective amount of ketamine, or a pharmaceutically acceptable salt thereof.

In some embodiments, the anti-seizure drug is selected from the group consisting of: topiramate, valproic acid, felbamate, rufinamide, stiripentol, fenfluramine, clobazam, clonazepam, lorazepam, gabapentin, pregabalin, retigabine, phenytoin, carbamazepine, oxcarbazepine, eslicarbazepine acetate, lamotrigine, lacosamide, zonisamide, levetiracetam, brivaracetam, ethosuximide, perampanel, phenobarbital, primidone, epidiolex, cenobamate, and tiagabine.

In some embodiments, after administering the ketamine the frequency, severity, and/or duration of one or more (e.g., two or more, three or more, four or more, two, three, or four) symptoms of the epilepsy is reduced. In this context, the reduction in frequency, severity, and/or duration of one or more symptoms can, for example, comprise reducing the frequency, severity, and/or duration of the symptoms when compared to (1) baseline, i.e., the frequency, severity, and/or duration of the one or more symptoms in the subject before start of the treatment (e.g., before administration of the ketamine, and wherein the frequency, severity, and/or duration of the one or more symptoms before administration of the one or more therapeutic agents can, for example, be evaluated by a single measurement or assessment, or an average of a plurality of measurements or assessments taken, e.g., over the course of a 1 month period, a 3 week period, 2 week period, a 7 day period, a 6 day period, a 5 day period, a 4 day period, a 3 day period, a 2 day period, or a 1 day period (e.g., a 7 day period)), wherein, for example, the reduction in frequency, severity, and/or duration of the symptoms is measured about 1 hour after treatment (e.g., after about 2 hours, 4 hours, 6 hours, 8 hours, 16 hours, 1 day, 2 days, 3 days, 4 days, 5 days, 6 days, 1 week, 1.5 weeks, 2 weeks, 3 weeks, 4 weeks, 1 month, 6 weeks, 2 months, 3 months, or 1 year of treatment); and/or (2) the frequency, severity, and/or duration of the one or more symptoms experienced by a subject after the subject was administered a placebo (e.g., a material, substance, or article that does not contain ketamine).

For example, the frequency of one or more symptoms of the epilepsy is reduced. For example, the severity of one or more symptoms of the epilepsy is reduced. For example, the duration of one or more symptoms of the epilepsy is reduced.

In some embodiments, the one or more symptoms of the epilepsy are selected from the group consisting of: focal seizures, generalized seizures, non-convulsive seizures, involuntary muscle contractions, auras, Jacksonian march, sensory disturbances, lip smacking, object lifting, involuntary vocalizations, erratic breathing, blue skin, loss of bladder or bowel control, tongue biting, crying, loss of consciousness, stumbling, falling, loss of balance, rapid eye blinking, catatonia, twitching, change in sensation (e.g., change in taste or smell), confusion, memory loss, or any combination thereof. In some embodiments, the one or more symptoms of the epilepsy are selected from the group consisting of: focal seizures, generalized seizures, non-convulsive seizures, involuntary muscle contractions, auras, Jacksonian march, sensory disturbances, lip smacking, object lifting, involuntary vocalizations, erratic breathing, blue skin, loss of bladder or bowel control, tongue biting, or any combination thereof.

In some embodiments, the focal seizure is a seizure of the left hemisphere of the brain. In some embodiments, the focal seizure is a seizure of the right hemisphere of the brain.

In some embodiments, the generalized seizures are selected from the group consisting of: tonic-clonic, tonic, clonic, myoclonic, absence, and atonic seizures.

In some embodiments, the subject experiences substantially no anesthesia after administration of the ketamine. In some embodiments, after administration of the ketamine, the subject talks (e.g., answers questions correctly), responds to stimuli (e.g., touch, pain, specific sounds, smells, and visual stimuli), and/or exhibits behaviors consistent with awareness and alertness.

In some embodiments, the therapeutically effective amount is a sub-anesthetic dose of ketamine. In some embodiments, the therapeutically effective amount is from about 2% to about 80% (e.g., about 2% to about 70%, about 2% to about 60%, about 2% to about 50%, about 2% to about 40%, about 2% to about 30%, about 2% to about 20%, about 2% to about 10%, about 10% to about 80%, about 20% to about 80%, about 30% to about 80%, about 40% to about 80%, about 50% to about 80%, about 60% to about 80%, about 10% to about 30%, about 10% to about 40%, about 10% to about 50%, about 5%, about 10%, about 15%, about 20%, about 25%, about 30%, about 35%, or about 40%) of the dose required to produce anesthesia in a subject.

In some embodiments, the subject receives a single dose of ketamine. In some embodiments, the subject receives multiple doses of ketamine at spaced apart intervals. In some embodiments, the ketamine is administered at least once per month (e.g., at least twice per month, at least three times per month, at least 4 times per month, at least once per week, at least twice per week, at least three times per week, at least four times per week, at least five times per week, at least 6 times per week, at least once daily, at least twice daily, at least three times daily, twice per month, three times per month, 4 times per month, once per week, twice per week, three times per week, four times per week, five times per week, 6 times per week, once daily, twice daily, or three times daily). In some embodiments, the ketamine is administered 3 times per week.

In some embodiments, the multiple doses of ketamine are administered over at least 1 day, for example, at least 2 days, at least 3 days, at least four days, at least five days, at least six days, at least 1 week, at least 2 weeks, at least 3 weeks, at least four weeks, at least 1 month, at least 2 months, at least 3 months, at least 6 months, at least 1 year, at least 2 years, at least 3 years, at least 5 years, at least 7 years, at least 10 years, at least 15 years, at least 20 years, or over the subject's lifespan. In some embodiments, the frequency, severity, and/or duration of the seizures is reduced after administering multiple doses of ketamine over up to 1 day, up to 2 days, up to 3 days, up to four days, up to five days, up to six days, up to 1 week, up to 2 weeks, up to 3 weeks, up to four weeks, up to 1 month, up to 2 months, up to 3 months, up to 6 months, or up to 1 year.

In some embodiments, the ketamine is administered three times per week for one week. In some embodiments, the ketamine is administered two times per week for one week. In some embodiments, the ketamine is administered once per week for one week. In some embodiments, the ketamine is administered three times per week for two weeks. In some embodiments, the ketamine is administered two times per week for two weeks. In some embodiments, the ketamine is administered once per week for two weeks. In some embodiments, the ketamine is administered three times per week for three weeks. In some embodiments, the ketamine is administered two times per week for three weeks. In some embodiments, the ketamine is administered once per week for three weeks. In some embodiments, the ketamine is administered three times per week for one month. In some embodiments, the ketamine is administered two times per week for one month. In some embodiments, the ketamine is administered once per week for one month. In some embodiments, the ketamine is administered three times per week for two months. In some embodiments, the ketamine is administered two times per week for two months. In some embodiments, the ketamine is administered once per week for two months. In some embodiments, the ketamine is administered three times per week for three months. In some embodiments, the ketamine is administered two times per week for three months. In some embodiments, the ketamine is administered once per week for three months.

In some embodiments, the frequency of administering the ketamine is decreased after an initial dosing period. In some embodiments, the ketamine is administered 2-3 times per week for about 1 month then 1-2 times per week for about 1 month. In some embodiments, the ketamine is administered 3 times per week for about 1 month then 2 times per week for about 1 month. In some embodiments, the ketamine is administered 2 times per week for about 1 month then once per week for about 1 month. In some embodiments, the ketamine is administered 2-3 times per week for about 1 month then 1-2 times per week for about 1 month then once per week afterwards. In some embodiments, the ketamine is administered 3 times per week for about 1 month then 2 times per week for about 1 month then once per week afterwards.

In some embodiments, the ketamine is administered during a seizure. In some embodiments, the ketamine is administered during a seizure, and the subject transitions to a postictal state or a normal state sooner than if the subject had not been administered ketamine. In some embodiments, the ketamine is administered during a seizure, and the subject transitions to a postictal state or a normal state (e.g., a normal state) within about 2 hours (e.g., about 1 hour and 45 minutes, about 1 hour and 30 minutes, about 1 hour and 15 minutes, about 1 hour, about 45 minutes, about 30 minutes, about 25 minutes, about 20 minutes, about 15 minutes, about 10 minutes, about 5 minutes, about 3 minutes, about 2 minutes, about 1 minute, or about 30 seconds) after administration of the ketamine. In some embodiments, the ketamine is administered during a seizure, and the subject transitions to a postictal state or a normal state within 10 minutes after administration of the ketamine. In some embodiments, the ketamine is administered during a seizure, and the subject transitions to a postictal state or a normal state within 5 minutes after administration of the ketamine.

In some embodiments, the ketamine is administered during a seizure, and the subject transitions to a postictal state within about 2 hours (e.g., about 1 hour and 45 minutes, about 1 hour and 30 minutes, about 1 hour and 15 minutes, about 1 hour, about 45 minutes, about 30 minutes, about 25 minutes, about 20 minutes, about 15 minutes, about 10 minutes, about 5 minutes, about 3 minutes, about 2 minutes, about 1 minute, or about 30 seconds) after administration of the ketamine. In some embodiments, the ketamine is administered during a seizure, and the subject transitions to a postictal state within 10 minutes after administration of the ketamine. In some embodiments, the ketamine is administered during a seizure, and the subject transitions to a postictal state within 5 minutes after administration of the ketamine.

In some embodiments, the ketamine is administered during a seizure, and the subject transitions to a normal state within about 2 hours (e.g., about 1 hour and 45 minutes, about 1 hour and 30 minutes, about 1 hour and 15 minutes, about 1 hour, about 45 minutes, about 30 minutes, about 25 minutes, about 20 minutes, about 15 minutes, about 10 minutes, about 5 minutes, about 3 minutes, about 2 minutes, about 1 minute, or about 30 seconds) after administration of the ketamine. In some embodiments, the ketamine is administered during a seizure, and the subject transitions to a normal state within 10 minutes after administration of the ketamine. In some embodiments, the ketamine is administered during a seizure, and the subject transitions to a normal state within 5 minutes after administration of the ketamine.

In some embodiments, the ketamine is esketamine (i.e., (S)-ketamine). In some embodiments, the ketamine is arketamine (i.e., (R)-ketamine). In some embodiments, the ketamine is esketamine or the R enantiomer of ketamine. In some embodiments, the ketamine is a mixture of esketamine and arketamine. In some embodiments, the mixture is enriched in esketamine. In some embodiments, the mixture is about 60% esketamine and about 40% arketamine. In some embodiments, the mixture is about 70% esketamine and about 30% arketamine. In some embodiments, the mixture is about 80% esketamine and about 20% arketamine. In some embodiments, the mixture is about 90% esketamine and about 10% arketamine. In some embodiments, the mixture is about 95% esketamine and about 5% arketamine. In some embodiments, the mixture is about 98% esketamine and about 2% arketamine.

In some embodiments, after administering the ketamine the frequency of seizures is reduced. In this context, the reduction in frequency of seizures can, for example, comprise reducing the frequency of seizures when compared to (1) baseline, i.e., the frequency of seizures in the subject before start of the treatment (e.g., before administration of the ketamine, and wherein the frequency of the seizures before administration of the ketamine can, for example, be evaluated by a single measurement or assessment, or an average of a plurality of measurements or assessments taken, e.g., over the course of a 1 month period, a 3 week period, 2 week period, a 7 day period, a 6 day period, a 5 day period, a 4 day period, a 3 day period, a 2 day period, or a 1 day period (e.g., a 7 day period)), wherein, for example, the reduction in frequency of the seizures is measured about 1 hour after treatment (e.g., after about 2 hours, 4 hours, 6 hours, 8 hours, 16 hours, 1 day, 2 days, 3 days, 4 days, 5 days, 6 days, 1 week, 1.5 weeks, 2 weeks, 3 weeks, 4 weeks, 1 month, 6 weeks, 2 months, 3 months, or 1 year of treatment); and/or (2) the frequency of seizures experienced by a subject after the subject was administered a placebo (e.g., a material, substance, or article that does not contain ketamine).

In some embodiments, the frequency of seizures is assessed by a seizure diary. In some embodiments, after administering the ketamine the frequency of seizures according to the seizure diary is reduced.

In some embodiments, the reduction in seizure frequency is at least about 2%, for example, at least about 4%, at least about 6%, at least about 8%, at least about 10%, at least about 12%, at least about 15%, at least about 20%, at least about 25%, at least about 30%, at least about 35%, at least about 40%, at least about 45%, at least about 50%, at least about 60%, at least about 70%, at least about 80%, at least about 90%, or seizures cease to occur in the subject. In some embodiments, the reduction in seizure frequency is at least about 10%. In some embodiments, the reduction in seizure frequency is at least about 25%. In some embodiments, the reduction in seizure frequency is at least about 50%.

In some embodiments, the subject is identified or diagnosed as having insomnia. In some embodiments, after administering the ketamine, average daily sleep duration is increased in the subject. Sleep duration can, for example, be measured by an increase in the average sleep duration per day by, e.g., an application, activity tracker, or smartwatch relative to average sleep per day before administration of the ketamine. The average can be an average of sleep over at least 2 days, for example, at least 3 days, 4 days, 5 days, 1 week, 9 days, 10 days, 2 weeks, 3 weeks, 4 weeks, 1 month, 2 months, or 3 months. The days used to calculate the averages fall within 1 week, 2 weeks, 1 month, 2 months, or 3 months of the first administration of the ketamine.

In some embodiments, the subject is identified or diagnosed as having cognitive impairment. In some embodiments, after administering the ketamine, one or more symptoms of the cognitive impairment of the subject are improved relative to before administration of the ketamine. This improvement can be measured by, e.g., the Mini-Mental State Examination (MMSE), the Montreal Cognitive Assessment, the Delayed three-word recall test, the Clock-drawing test, serum electrolyte concentration, or serum calcium concentration. In some embodiments, the symptoms of cognitive impairment include, but are not limited to poor memory, difficulty learning new information, difficulty concentrating, or poor decision-making ability.

In some embodiments, the subject is identified or diagnosed as having migraines. In some embodiments, after administering the ketamine, the subject experiences less frequent, less severe, and/or shorter duration migraine attacks and/or migraine symptoms relative to before administration of the ketamine. In some embodiments, the migraine symptoms include, but are not limited to pain in the face, pain in the neck, throbbing pain, sensitivity to light, distorted vision, seeing flashes of light, dizziness, lightheadedness, malaise, sensitivity to sound, nausea, vomiting, irritability, nasal congestion, or scalp tenderness.

In some embodiments, the subject was identified or diagnosed as having depression and/or anxiety before administering the ketamine. In some embodiments, after administration of the ketamine, the depression and/or anxiety are reduced. In this context, reducing the depression and/or anxiety in the subject can, for example, comprise reducing the depression and/or anxiety when compared to (1) baseline, i.e., the depression and/or anxiety in the subject before start of the treatment (e.g., before administration of the ketamine, and wherein the depression and/or anxiety before administration of the ketamine can, for example, be evaluated by a single measurement or assessment, or an average of a plurality of measurements or assessments taken, e.g., over the course of a 1 month period, a 3 week period, 2 week period, a 7 day period, a 6 day period, a 5 day period, a 4 day period, a 3 day period, a 2 day period, or a 1 day period (e.g., a 7 day period)), wherein, for example, the reduction in depression and/or anxiety is measured about 1 hour after treatment (e.g., after about 2 hours, 4 hours, 6 hours, 8 hours, 16 hours, 1 day, 2 days, 3 days, 4 days, 5 days, 6 days, 1 week, 1.5 weeks, 2 weeks, 3 weeks, 4 weeks, 1 month, 6 weeks, 2 months, 3 months, or 1 year of treatment); and/or (2) the depression and/or anxiety experienced by a subject after the subject was administered a placebo (e.g., a material, substance, or article that does not contain ketamine). In some embodiments, the depression includes but is not limited to any of: major depressive disorder, treatment resistant depression, single episode, recurrent major depressive disorder-unipolar depression, seasonal affective disorder-winter depression, bipolar mood disorder-bipolar depression, mood disorder due to a general medical condition with major depressive-like episode, or mood disorder due to a general medical condition with depressive features.

A means of evaluating depression in subjects suffering from epilepsy is the Neurological Disorders Depression Inventory for Epilepsy (NDDI-E). Further information about the NDDI-E can be found in Gill S et al. Epilepsia 2017, 58(5), 695-705, which is incorporated by reference herein in its entirety. In some embodiments, the Neurological Disorders Depression Inventory for Epilepsy (NDDI-E) score of the subject is lower after administering the ketamine. In this context, the NDDI-E score of the subject is lower after administering the ketamine than before administering the ketamine. In some embodiments, the NDDI-E score is at least 0.5 points lower (e.g., at least 1 point lower, at least 2 points lower, at least 3 points lower, at least 4 points lower, at least 5 points lower, at least 6 points lower, at least 7 points lower, at least 8 points lower, 1 point lower, 2 points lower, 3 points lower, 4 points lower, 5 points lower, 6 points lower, 7 points lower, or 8 points lower) after administering the ketamine. In some embodiments, the NDDI-E score is at least 1 point lower after administering the ketamine. In some embodiments, the NDDI-E score is at least 3 points lower after administering the ketamine. In some embodiments, the NDDI-E score before administering the ketamine is greater than 16, and the NDDI-E score after administering the ketamine is less than or equal to (e.g., less than) 16. In some embodiments, the NDDI-E score before administering the ketamine is greater than 16, and the NDDI-E score after administering the ketamine is less than or equal to (e.g., less than) 11. In some embodiments, the NDDI-E score before administering the ketamine is greater than 11, and the NDDI-E score after administering the ketamine is less than or equal to (e.g., less than) 11.

A means of evaluating generalized anxiety disorder is the Generalized Anxiety Disorder-7 (GAD-7). Further information about the GAD-7 can be found in Spitzer et. al. Arch Intern Med. 2006, 166(10), 1092-1097, which is incorporated by reference herein in its entirety. In some embodiments, the Generalized Anxiety Disorder 7 (GAD-7) score of the subject is lower after administering the ketamine. In this context, the GAD-7 score of the subject is lower after administering the ketamine than before administering the ketamine. In some embodiments, the GAD-7 score is at least 5% lower (e.g., at least 8% lower, at least 10% lower, at least 15% lower, at least 20% lower, at least 25% lower, at least 30% lower, at least 40% lower, at least 50% lower, at least 70% lower or at least 90% lower) after administering the ketamine. In some embodiments, the GAD-7 score is at least 10% lower after administering the ketamine. In some embodiments, the GAD-7 score is at least 30% points lower after administering the ketamine. In some embodiments, the GAD-7 score of the subject before administering the ketamine is 15 or greater and the GAD-7 score of the subject after administering the ketamine is 10 or less than 10. In some embodiments, the GAD-7 score of the subject before administering the ketamine is 15 or greater and the GAD-7 score of the subject after administering the ketamine is 5 or less than 5. In some embodiments, the GAD-7 score of the subject before administering the ketamine is 15 or greater and the GAD-7 score of the subject after administering the ketamine is 10 or less than 10. In some embodiments, the GAD-7 score of the subject before administering the ketamine is 10 or greater and the GAD-7 score of the subject after administering the ketamine is 5 or less than 5. In some embodiments, the GAD-7 score of the subject before administering the ketamine is 15 and the GAD-7 score of the subject after administering the ketamine is less than 5.

A means of evaluating anxiety, depression, and mood among subjects with mental retardation is the Anxiety, Depression and Mood Scale (ADAMS). Further information about the ADAMS can be found in Esbensen A, et al. J. Autism Dev. Disord. 2003, 33(6), 617-629, which is incorporated by reference herein in its entirety. In some embodiments, the Anxiety, Depression and Mood Scale (ADAMS) score of the subject is lower after administering the ketamine. In this context, the ADAMS score of the subject is lower after administering the ketamine than before administering the ketamine. In some embodiments, the ADAMS score is at least 5% lower (e.g., at least 8% lower, at least 10% lower, at least 15% lower, at least 20% lower, at least 25% lower, at least 30% lower, at least 40% lower, at least 50% lower, at least 70% lower or at least 90% lower) after administering the ketamine. In some embodiments, the ADAMS score is at least 10% lower after administering the ketamine. In some embodiments, the ADAMS score is at least 30% points lower after administering the ketamine.

A means of evaluating quality of life of subjects who have epilepsy is the Quality of Life in Epilepsy Inventory-10 (QOLIE-10). Further information about the QOLIE-10 can be found in Gill S. et al. Epilepsia 2017, 58(5), 695-705, which is incorporated by reference herein in its entirety. In some embodiments, the Generalized Anxiety Disorder 7 (QOLIE-10) score of the subject is lower after administering the ketamine. In this context, the QOLIE-10 score of the subject is lower after administering the ketamine than before administering the ketamine. In some embodiments, the QOLIE-10 score is at least 5% lower (e.g., at least 8% lower, at least 10% lower, at least 15% lower, at least 20% lower, at least 25% lower, at least 30% lower, at least 40% lower, at least 50% lower, at least 70% lower or at least 90% lower) after administering the ketamine. In some embodiments, the QOLIE-10 score is at least 10% lower after administering the ketamine. In some embodiments, the QOLIE-10 score is at least 30% points lower after administering the ketamine.

In some embodiments, the subject is age 2 or older (e.g., age 4, 6, 8, 10, 12, 14, 16, 18, 21, 25, 30, 35, 40, 45, 50, 55, 60, 65, 70, 75, or 80 or older). In some embodiments, the subject is age 80 or younger (e.g., age 75, 70, 65, 60, 55, 50, 45, 40, 35, 30, 25, 21, 18, 16, 14, 12, 10,8,5,4, or 2 or younger).

In some embodiments, the subject is identified or diagnosed as having symptomatic epilepsy.

In some embodiments, the clinical record of the subject shows findings of abnormal neuroimaging.

In some embodiments, the clinical record of the subject shows an abnormal electroencephalogram (EEG).

In some embodiments, the subject is or was identified or diagnosed as having mental retardation.

In some embodiments, the subject is identified or diagnosed as having a neuropsyhiatric disorder.

In some embodiments, the clinical record of the subject shows that the subject had a febrile seizure.

In some embodiments, the subject is identified or diagnosed as having status epilepticus.

In some embodiments, the subject has any of the diseases, disorders, or medical history correlated with drug-resistant epilepsy disclosed in Kalilani L, et. al. Epilepsia. 2018, 59(12), 2179-2193, which is incorporated by reference herein in its entirety.

In some embodiments, the clinical record of the subject indicates a gut microbiome characteristic of microbial imbalance. For example, the gastrointestinal tract of the subject contains a high population of harmful bacteria such as Staphylococcus species or Clostridium species, or an abnormally low population of beneficial bacteria such as Lactobacillus species and/or Bifidobacterium species. Further information on the relationship between gut microbiome and drug-resistant epilepsy can be found in Holmes, M. et. al. Epilepsia, 2020, 61(12), 2619-2628, which is incorporated by reference herein in its entirety.

In some embodiments, the subject is identified or diagnosed as having a cardiovascular disorder. In some embodiments, the cardiovascular disorder is ischemic heart disease.

In some embodiments, the subject is identified or diagnosed as having peptic ulcers.

In some embodiments, the subject is identified or diagnosed as having arthritis (e.g., osteoarthritis or rheumatoid arthritis).

In some embodiments, the method comprises administering a second therapeutic agent. In some embodiments, the second therapeutic agent is an anti-seizure drug, an antidepressant, an anti-psychotic, an NMDA antagonist, or a drug that ameliorates or exacerbates oxidative stress disorder.

In some embodiments, the second therapeutic agent is an anti-seizure drug. In some embodiments, the anti-seizure drug is selected from the group consisting of: topiramate, valproic acid, felbamate, rufinamide, stiripentol, fenfluramine, clobazam, clonazepam, lorazepam, gabapentin, pregabalin, retigabine, phenytoin, carbamazepine, oxcarbazepine, eslicarbazepine acetate, lamotrigine, lacosamide, zonisamide, levetiracetam, brivaracetam, ethosuximide, perampanel, phenobarbital, primidone, epidiolex, cenobamate, and tiagabine.

In some embodiments, the second therapeutic agent is an antidepressant. In some embodiments, the antidepressant is selected from the group consisting of: lithium salts, carbamazepine, valproic acid, lysergic acid diethylamide (LSD), p-chlorophenylalanine, p-propyidopacetamide dithiocarbamate derivatives (e.g., FLA63); anti-anxiety drugs (e.g., diazepam; monoamine oxidase (MAO) inhibitors, e.g., iproniazid, clorgyline, phenelzine, tranylcypromine, and isocarboxazid; biogenic amine uptake blockers, e.g., tricyclic antidepressants such as desipramine, imipramine and amitriptyline; atypical antidepressants such as mirtazapine, nefazodone, bupropion; serotonin reuptake inhibitors e.g., fluoxetine, venlafaxine, and duloxetine.

In some embodiments, the second therapeutic agent is an anti-psychotic. In some embodiments, the anti-psychotic is phenothiazine derivatives (e.g., chlorpromazine (thorazine) and trifluopromazine)), butyrophenones (e.g., haloperidol (Haldol)), thioxanthene derivatives (e.g., chlorprothixene), sand dibenzodiazepines (e.g., clozapine); benzodiazepines; dopaminergic agonists and antagonists e.g., L-DOPA, cocaine, amphetamine, α-methyl-tyrosine, reserpine, tetrabenazine, benztropine, pargyline; noradrenergic agonists and antagonists, e.g., clonidine, phenoxybenzamine, phentolamine, tropolone.

In some embodiments, the second therapeutic agent is an NMDA antagonist. In some embodiments, the NMDA antagonist is selected from the group consisting of: pethidine, levorphanol, methadone, dextropropoxyphene, tramadol, ketobemidone, dextromethorphan (DXM), phencyclidine (PCP), methoxetamine (MXE), and nitrous oxide.

In some embodiments, the second therapeutic agent is a drug that ameliorates or exacerbates oxidative stress disorder. In some embodiments, the drug that ameliorates or exacerbates oxidative stress disorder is reduced IS glutathione (GSH), glutathione precursors, e.g., N-acetylcysteine; antioxidants, e.g., vitamins E and C, beta carotene and quinones; inhibitors of lipid membrane peroxidation, e.g., 21-aminosteroid U74006F (tirilazad mesylate), and lazaroids; antioxidants such as mazindol; 2c dizocilpine maleate; selegiline; sulfhydryls N-acetylcysteine and cysteamine; dimethylthiourea; EUK-8 (which is a synthetic, low molecular salen-manganese complex); synthetic manganese-based metalloprotein superoxide dismutase mimic, SC52608; free radical scavengers or suppressors, e.g., pegorgotein, tocotrienol, tocopheral, MDL7418, LY231617, MCI-186, AVS (nicaraven), allopurinol, rifampicin, oxypurinol, hypochlorous acid, or recombinant human Cu, Zn-SOD.

It is understood that the peak ketamine plasma level (i.e., the Cₘₐₓ of ketamine) is the highest plasma concentration of the ketamine observed in the subject after administration of the ketamine. In some embodiments peak ketamine plasma level (i.e., the Cₘₐₓ of ketamine) is from about 0.1 ng/mL to about 2000 ng/mL. For example, from about 0.1 ng/mL to about 2000 ng/mL, from about 0.1 ng/mL to about 1500 ng/mL, from about 0.1 ng/mL to about 1200 ng/mL, from about 0.1 ng/mL to about 1000 ng/mL, from about 0.1 ng/mL to about 800 ng/mL, from about 0.1 ng/mL to about 600 ng/mL, from about 0.1 ng/mL to about 500 ng/mL, from about 0.1 ng/mL to about 400 ng/mL, from about 0.1 ng/mL to about 300 ng/mL, from about 0.1 ng/mL to about 200 ng/mL, from about 0.1 ng/mL to about 100 ng/mL, from about 0.1 ng/mL to about 70 ng/mL, from about 0.1 ng/mL to about 50 ng/mL, from about 0.1 ng/mL to about 40 ng/mL, from about 0.1 ng/mL to about 30 ng/mL, from about 0.1 ng/mL to about 20 ng/mL, from about 0.1 ng/mL to about 10 ng/mL, from about 0.1 ng/mL to about 5 ng/mL, from about 20 ng/mL to about 50 ng/mL, from about 25 ng/mL to about 35 ng/mL, from about 28 ng/mL to about 32 ng/mL, from about 200 ng/mL to about 800 ng/mL, from about 300 ng/mL to about 700 ng/mL, from about 400 ng/mL to about 600 ng/mL, from about 450 ng/mL to about 550 ng/mL, from about 480 ng/mL to about 520 ng/mL, about 10 ng/mL, about 20 ng/mL, about 30 ng/mL, about 40 ng/mL, about 50 ng/mL, about 100 ng/mL, about 200 ng/mL, about 300 ng/mL, about 400 ng/mL, about 500 ng/mL, about 600 ng/mL, or about 700 ng/mL.

In some embodiments, the subject is human.

In some embodiments, the subject is not pregnant.

In some embodiments, the subject is not hypertensive.

In some embodiments, the subject is not afflicted with heart failure.

In some embodiments, the subject is not afflicted with a cardiovascular disease.

In some embodiments, the subject has not previously had a stroke.

In some embodiments, the subject is not afflicted with brain trauma.

In some embodiments, the subject is not afflicted with cerebral edema.

In some embodiments, the subject is not afflicted with an intracerebral hemorrhage.

In some embodiments, the subject is not afflicted with hyperthyroidism (e.g., uncontrolled hyperthyroidism).

In some embodiments, the subject is not afflicted with thyrotoxic crisis.

### Dosage, Routes of Administration, and Pharmaceutical Compositions

In some embodiments, a method of treating DRE as provided herein includes treating a subject (e.g., a human) suffering from DRE with a therapeutically effective amount of ketamine (e.g., esketamine). The actual dose will vary, depending on the body weight of the patient, the patients response to treatment, the severity of the epilepsy, the route of administration, the nature of medications administered concurrently, the number of doses to be administered per day, and other factors generally considered by the ordinary skilled physician in the administration of drugs. In some embodiments, the therapeutically effective amount of ketamine is a dose of about 0.01 to about 2.0 mg of ketamine per kilogram of body weight of the patient (mg/kg) to treat DRE. In some embodiments, the dose is about 0.1 to about 2.0 mg/kg of ketamine. In some embodiments, the dose is about 0.05 to about 0.5 mg/kg of ketamine. In some embodiments, the dose is about 0.5 to about 1.5 mg/kg of ketamine. In some embodiments, the dose is about 0.7 to about 1.7 mg/kg of ketamine. In some embodiments, the dose is less than about 0.5 mg/kg, less that about 0.4 mg/kg or less than about 0.3 mg/kg of ketamine. In some embodiments, the therapeutically effective amount of ketamine is a dose in the range of from about 0.01 mg/kg to about 2.0 mg/kg. In some embodiments, the therapeutically effective amount of ketamine is a dose in the range of from about 0.01 mg/kg to about 1.5 mg/kg. In some embodiments, the therapeutically effective amount of ketamine is a dose in the range of from about 0.01 mg/kg to about 1 mg/kg. In some embodiments, the therapeutically effective amount of ketamine is a dose in the range of from about 0.01 mg/kg to about 0.75 mg/kg. In some embodiments, the therapeutically effective amount of ketamine is a dose in the range of from about 0.75 mg/kg to about 1.5 mg/kg. In some embodiments, the therapeutically effective amount of ketamine is a dose in the range of from about 0.5 mg/kg to about 1.2 mg/kg. In some embodiments, the therapeutically effective amount of ketamine is a dose in the range of from about 0.05 mg/kg to about 0.5 mg/kg. In some embodiments, the therapeutically effective amount of ketamine is a dose in the range of from about 0.1 mg/kg to about 0.9 mg/kg. In some embodiments, the therapeutically effective amount of ketamine is a dose in the range of from about 0.2 mg/kg to about 0.9 mg/kg. In some embodiments, the therapeutically effective amount of ketamine is a dose in the range of from about 0.3 mg/kg to about 0.8 mg/kg. In some embodiments, the therapeutically effective amount of ketamine is a dose of about 0.2 mg/kg or in an amount of about 0.4 mg/kg. In some embodiments, the therapeutically effective amount of ketamine is about 0.5 ml/kg.

In some embodiments, the therapeutically effective amount of ketamine is a sub-anesthetic amount of ketamine for the individual. In some embodiments, the individual is treated with ketamine via intravenous or intranasal administration. In some embodiments, the ketamine is administered intravenously. In some embodiments, the ketamine is administered intranasally. In some embodiments, the ketamine is contacted with the nasal mucosa. In some embodiments, the individual is treated intranasally with ketamine, substantially only via the nasal respiratory epithelium, compared to treatment via the nasal olfactory epithelium (for example, at least 80%, at least 85%, at least 90%, at least 95%, at least 98%, or at least 99% of the ketamine is delivered via the nasal respiratory epithelium, compared to treatment via the nasal olfactory epithelium). In some embodiments, the individual is treated intranasally with ketamine, substantially only via the nasal olfactory epithelium, compared to treatment via the nasal respiratory epithelium (for example, at least 80%, at least 85%, at least 90%, at least 95%, at least 98%, or at least 99% of the ketamine is delivered vis the nasal olfactory epithelium, compared to treatment via the nasal respiratory epithelium). In some embodiments, the individual is treated with a single dose of the therapeutically effective amount of ketamine. In some embodiments, the individual is treated with multiple doses of the therapeutically effective amount of ketamine. In some embodiments, after an initial treatment of one or more doses of ketamine, the subject is subsequently treated with one or more higher doses of ketamine.

In some embodiments, the one or more higher doses are determined based on the response of the subject to the initial treatment dose(s). In some embodiments, a further adjustment of the ketamine dose may be made, with the objective of achieving a balance between treating the ketamine symptoms and minimization of any adverse or undesirable effects of the ketamine.

In some embodiments, the total dose of ketamine (e.g., esketamine) is about 25 mg. In some embodiments, the total dose of esketamine is about 50 mg. In some embodiments, the total dose of esketamine is about 75 mg. In some embodiments, the total dose of esketamine is about 100 mg. In some embodiments, the total dose of esketamine is about 1.1 mg/kg. In some embodiments, the total dose of esketamine is about 1.2 mg/kg 1.3 mg/kg, 1.4 mg/kg, 1.5 mg/kg, 1.6 mg/kg, 1.7 mg/kg, 1.8 mg/kg, 1.9 mg/kg, or 2.0 mg/kg.

In any of the above aspects, the ketamine can be esketamine or R-ketamine. Thus, also provided is a method of treating DRE comprising administering to a patient in need of such treatment an effective amount for treating DRE of ketamine, R ketamine or esketamine. In some embodiments, the esketamine or R ketamine is administered intravenously. In some embodiments, the esketamine or R ketamine is administered intranasally.

Also provided herein is a method of treating DRE that includes treating a subject (e.g., a human) suffering from DRE with a therapeutically effective amount of esketamine. In some embodiments, the esketamine is administered in an amount in the range of from about 0.01 mg/kg to about 2.0 mg/kg. In some embodiments, the esketamine is administered in an amount in the range of from about 0.01 mg/kg to about 1.5 mg/kg. In some embodiments, the esketamine is administered in an amount in the range of from about 0.01 mg/kg to about 1 mg/kg. In some embodiments, the esketamine is administered in an amount in the range of from about 0.01 mg/kg to about 0.75 mg/kg. In some embodiments, the esketamine is administered in an amount in the range of from about 0.75 mg/kg to about 1.5 mg/kg. In some embodiments, the esketamine is administered in an amount in the range of from about 0.5 mg/kg to about 1.2 mg/kg. In some embodiments, the esketamine is administered in an amount in the range of from about 0.05 mg/kg to about 0.5 mg/kg. In some embodiments, the esketamine is administered in an amount of about 0.2 mg/kg or in an amount of about 0.4 mg/kg.

In some embodiments, the total dose of esketamine is about 25 mg. In some embodiments, the total dose of esketamine is about 50 mg. In some embodiments, the total dose of esketamine is about 75 mg. In some embodiments, the total dose of esketamine is about 100 mg. In some embodiments, the total dose of esketamine is about 1.1 mg/kg. In some embodiments, the total dose of esketamine is about 1.2 mg/kg 1.3 mg/kg, 1.4 mg/kg, 1.5 mg/kg, 1.6 mg/kg, 1.7 mg/kg, 1.8 mg/kg, 1.9 mg/kg, or 2.0 mg/kg.

In some embodiments, the esketamine is administered intravenously. In some embodiments, the esketamine is administered intranasally.

Also provided herein is a pharmaceutical composition that comprises ketamine (e.g., esketamine) and a pharmaceutically acceptable carrier, excipient or diluent, for use in treatment of DRE. The carrier is a macromolecule which is soluble in the circulatory system and which is physiologically acceptable where physiological acceptance means that those of skill in the art would accept injection of said carrier into a patient as part of a therapeutic regime. The carrier preferably is relatively stable in the circulatory system with an acceptable plasma half-life for clearance. Such macromolecules include but are not limited to soya lecithin, oleic acid and sorbitan trioleate, with sorbitan trioleate preferred. In some embodiments, the pharmaceutical composition is for intranasal or intravenous administration. In some embodiments, the pharmaceutical composition is for use in a method of treating DRE in a subject. In some embodiments, the ketamine is formulated with a pharmaceutically acceptable carrier or diluent. In some embodiments, the ketamine is formulated as a solution or suspension. In some embodiments, the carrier or diluent is aqueous. In some embodiments, the carrier or diluent comprises sterile phosphate buffered saline solution, bacteriostatic water, aqueous glycine, or any combination thereof.

Ketamine ((2-(2-chlorophenyl)-2-(methylamino)-cyclohexanone) is a general anesthetic used by anesthesiologists, veterinarians, and researchers. Pharmacologically, ketamine is a noncompetitive NMDA receptor (NMDAR) antagonist. More specifically, ketamine binds to the allosteric site of the NMDA receptor, effectively inhibiting its channel. At high, fully anesthetic level doses, ketamine has also been found to bind to μ-opioid receptors type 2 in cultured human neuroblastoma cells—however, without agonist activity—and to sigma receptors in rats. Also, ketamine interacts with muscarinic receptors, descending monoaminergic pain pathways and voltage-gated calcium channels.

Ketamine is a chiral compound. The S(+) and R(−) stereoisomers bind NMDA receptors with different affinities: Ki=3200 and 1100 nM, respectively. Vranken et al. studied the use of an iontophoretic patch (a mechanism of delivery in which the electrically charged drug is transmitted by pulses of galvanic current) in 33 men and women in an investigation that studied the use of an iontophoretic patch to deliver ketamine for the treatment of intractable central neuropathic pain. S(+)-ketamine (also referred to as “(S)-ketamine” or “esketamine”) was found to be two times more potent than racemic mixture of ketamine. Most pharmaceutical preparations of ketamine are racemic; however, some brands reportedly have (mostly undocumented) differences in enantiomeric proportions. The more active (S)-ketamine enantiomer is available for medical use under the brand name Ketanest S. Its hydrochloride salt is sold as Ketanest, Ketaset, and Ketalar. See, Paul et al., “Comparison of racemic ketamine and S-ketamine in treatment-resistant major depression: report of two cases”, World J. of Bio. Psych., 2009, pp 241-244, Vol. 10(3) describe two cases studies in which patients with a history of recurrent major depression were treated with intravenous infusion of ketamine and S-ketamine; Paskalis et al., “Oral Administration of the NMDA Receptor Antagonist S-Ketamine as Add-on Therapy of Depression: A Case Series”, Pharmacopsychiatry, 2010, pp 33-35, Vol. 40 present four case studies where depressed patients received 1.25 mg/kg oral S-ketamine as add-on to standard antidepressant therapy; Noppers et al., “Absence of long-term analgesic effect from a short-term S-ketamine infusion on fibromyalgia pain: A randomized, prospective, double blind, active placebo-controlled trial”, Eur. J. of Pain., 2011, article in press, describe a trial assessing the analgesic efficacy of S-(+)-ketamine on fibromyalgia pain; Matthews et al., “Ketamine for Treatment-Resistant Unipolar Depression”, CNS Drugs, 2012, pp 1-16, provide a review of emerging literature on ketamine and a review of the pharmacology of both ketamine and S-ketamine; and International Patent Publication No. WO2013138322. As used herein, “ketamine” includes preparations of ketamine that contain a racemic mixture of S(+) and R(−) stereoisomers of ketamine, preparations that contain differences in the enantiomeric proportions of the S(+) and R(−) stereoisomers, and preparations that contain only one of the enantiomers (e.g., only S(+) ketamine or only R(−) ketamine). Intranasal ketamine is available under the brand name Spravato.

Intravenous administration of ketamine has been used for the rapid treatment of treatment-resistant major depression. A 0.5 mg/kg intravenous infusion given over 40 minutes resulted in improvements in depression within 2 hours post-injection; and continued for up to 1 week. There were no serious adverse events. Zarate et al., Am J Psychiatry, 2006, 163:153-5. Intranasal (IN) ketamine plasma levels used for treatment of pain are 3-4 fold lower than the intravenous (IV) ketamine studies in depression. The slow infusion of ketamine produces gradually increasing plasma levels during the infusion period. In some embodiments, these studies inform the dosing that can be used in the treatment of drug-resistant epilepsy.

A typical ketamine dose for induction of anesthesia for surgical procedures is between 1.0-2.0 mg/kg, with additional ketamine used to sustain anesthesia. In anesthesia, the target ketamine blood level is reached with ketamine bolus doses between 0.2-0.26 mg/kg over 1 min. The dose for ketamine plasma levels to produce DRE responses as opposed to the levels needed to produce anesthesia is in the range of 0.5 mg/kg over 40 min. The reports of dissociation in pain studies were significantly lower than the IV studies in major depressive disorder because the ketamine levels achieved intranasally in these studies were much lower. The intranasal dose used for pain (50 mg) is roughly equivalent to 0.1 mg/kg i.v. of ketamine. Such an administration may be administered over a 1 hour time period or more or less. It is expected that chronic administration of the intranasal formulation may be employed as necessary, ranging from daily to weekly, depending on response. In some embodiments, should the 50 mg intranasal dosage prove to be inadequate to treat epilepsy, effectively increasing doses, e.g., approximately 100 mg, approximately 150 mg, approximately 200 mg, approximately 250 mg total ketamine will be administered intranasally, to establish the relative equivalent of the about 0.5 mg/kg dosage usage in the IV studies. The intranasal formulation may eliminate the necessity of patient presentation to a hospital or clinic for intravenous administration. The subject can take intranasal ketamine in their own home, with no need for a needle stick. Thus, the acceptability of the treatment for patients will be better than with the IV ketamine. The patient may be one that is at least a moderately treatment-resistant patient, who is seeking new options for the rapid and safe reduction of epilepsy symptoms. The physician can monitor the subject as an outpatient, and could adjust dosage as they would for an orally administered medication.

In some embodiments, the DRE-alleviating dose of ketamine is approximately 0.01 to approximately 3 mg/kg of body weight, approximately 0.01 to approximately 2 mg/kg of body weight, approximately 0.01 to approximately 1.5 mg/kg of body weight, approximately 0.05 to approximately 1.4 mg/kg of body weight, approximately 0.05 to approximately 1.3 mg/kg of body weight, approximately 0.05 to approximately 1.2 mg/kg of body weight, approximately 0.05 to approximately 1.1 mg/kg of body weight, approximately 0.01 to approximately 1 mg/kg of body weight, or approximately 0.05 to approximately 0.7 mg/kg of body weight.

In some embodiments, the DRE-alleviating dose of esketamine is approximately 0.01 to approximately 3 mg/kg of body weight, approximately 0.01 to approximately 2 mg/kg of body weight, approximately 0.01 to approximately 1.5 mg/kg of body weight, approximately 0.05 to approximately 1.4 mg/kg of body weight, approximately 0.05 to approximately 1.3 mg/kg of body weight, approximately 0.05 to approximately 1.2 mg/kg of body weight, approximately 0.05 to approximately 1.1 mg/kg of body weight, approximately 0.01 to approximately 1 mg/kg of body weight, or approximately 0.05 to approximately 0.7 mg/kg of body weight.

In some embodiments, the DRE-alleviating dose of ketamine is approximately 0.01 mg to about 1000 mg, or any amount or range therein, preferably from about 0.01 mg to about 500 mg, or any amount or range therein, preferably from about 0.1 mg to about 250 mg, or any amount or range therein. In another aspect, the DRE-alleviating dose of ketamine is, e.g., 0.01 mg, 0.025 mg, 0.05 mg, 0.1 mg, 0.5 mg, 1 mg, 5 mg, 10 mg, 25 mg, 50 mg, 55 mg, 60 mg, 65 mg, 70 mg, 75 mg, 80 mg, 90 mg, 95 mg, 100 mg, 150 mg, 200 mg, 250 mg, or 500 mg.

In some embodiments, the DRE-alleviating dose of ketamine is approximately 0.01 mg to about 1000 mg, or any amount or range therein, preferably from about 0.01 mg to about 500 mg, or any amount or range therein, preferably from about 0.1 mg to about 250 mg, or any amount or range therein. In another aspect, the DRE-alleviating dose of ketamine is, e.g., 0.01 mg, 0.025 mg, 0.05 mg, 0.1 mg, 0.5 mg, 1 mg, 5 mg, 10 mg, 25 mg, 50 mg, 55 mg, 60 mg, 65 mg, 70 mg, 75 mg, 80 mg, 90 mg, 95 mg, 100 mg, 150 mg, 200 mg, 250 mg, or 500 mg.

In one aspect, the DRE-alleviating dose of esketamine is approximately 0.01 mg to about 1000 mg, or any amount or range therein, preferably from about 0.01 mg to about 500 mg, or any amount or range therein, preferably from about 0.1 mg to about 250 mg, or any amount or range therein. In another aspect, the DRE-alleviating dose of esketamine is, e.g., 0.01 mg, 0.025 mg, 0.05 mg, 0.1 mg, 0.5 mg, 1 mg, 5 mg, 10 mg, 25 mg, 50 mg, 55 mg, 60 mg, 65 mg, 70 mg, 75 mg, 80 mg, 90 mg, 95 mg, 100 mg, 150 mg, 200 mg, 250 mg, or 500 mg.

While it is possible to use a composition disclosed herein (e.g., a composition comprising ketamine) for therapy as is, it may be preferable to formulate the composition in a pharmaceutical formulation, e.g., in admixture with a suitable pharmaceutical excipient, diluent, or carrier selected with regard to the intended route of administration and standard pharmaceutical practice. Suitable carriers and their formulations are described in Remington: The Science and Practice of Pharmacy, 21^{st} ed., 2005, Lippincott, Williams & Wilkins, Phila., PA. Accordingly, in one aspect, a pharmaceutical composition or formulation comprises at least one active composition of ketamine in association with a pharmaceutically acceptable excipient, diluent, and/or carrier. The excipient, diluent and/or carrier must be “acceptable” in the sense of being compatible with the other ingredients of the formulation and not deleterious to the recipient thereof.

For in vivo administration to humans, the compositions can be formulated according to known methods used to prepare pharmaceutically useful compositions. Compositions may be designed to be short-acting, fast-releasing, long-acting, or sustained-releasing. Thus, pharmaceutical formulations may also be formulated for controlled release or for slow release.

When formulated in a pharmaceutical composition or formulation, ketamine can be admixed with a pharmaceutically acceptable carrier or excipient. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Other exemplary carriers include, but are not limited to, any of a number of standard pharmaceutical carriers such as sterile phosphate buffered saline solutions, bacteriostatic water, and the like. A variety of aqueous carriers may be used, e.g., water, buffered water, 0.4% saline, 0.3% glycine and the like.

The compositions and formulations described herein may be for administration by oral (solid or liquid), parenteral (intramuscular, intraperitoneal, intravenous (IV) or subcutaneous injection), transdermal (either passively or using ionophoresis or electroporation), transmucosal (nasal, intranasal, vaginal, rectal, or sublingual), or inhalation routes of administration, or using bioerodible inserts and can be formulated in dosage forms appropriate for each route of administration. The most suitable route in any given case will depend on the particular host, and nature and severity of the conditions for which the active ingredient is being administered. The compositions may be conveniently presented in unit dosage form and prepared by any of the methods well known in the art of pharmacy, and using well known carriers and excipients.

In general, preparations according to the present disclosure include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also optionally contain adjuvants, preserving, wetting, emulsifying, and dispersing agents. The pharmaceutical compositions may be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

A preferred route of administration of ketamine is intravenous (IV). Ketamine may thus also be prepared in a formulation or pharmaceutical composition appropriate for IV administration. Ketamine can be admixed with a pharmaceutically acceptable carrier or excipient as described above. By way of example, ketamine can be formulated in a saline solution for intravenous administration.

Another preferred mode of administration is intranasal administration, i.e., through the nasal mucosa and through the nose-brain pathway directly into the cerebrospinal fluid. Ming Wen, Discov Med, “Olfactory Targeting Through Intranasal Delivery of Biopharmaceutical Drugs to the Brain—Current Development,” 2011, 11:497-503, is hereby incorporated by reference in its entirety. As discussed in Wen, drugs administered intranasally may reach the brain via alternatives pathways. In one pathway, drugs, e.g., ketamine, are absorbed systemically, following absorption through the blood vessels of the nasal respiratory epithelium. Drugs delivered via this systemic pathway must first cross the blood brain barrier, prior to reaching the brain. In an alternative delivery pathway, drugs administered intranasally can be rapidly transported into the CNS via the connection between the olfactory epithelium at the roof of the nasal cavity and the trigeminal system of the brain. This affords a direct connection, with no synapse between the olfactory neurons and the brain. The pathway thus allows transport of active agents to the brain without passage through the blood brain barrier.

Excipients that may improve intranasal administration of ketamine include mucoadhesives (e.g., carbopol, carboxymethylcellulose, and hyaluronan), penetration enhancers that improve permeability and bioavailability of ketamine upon contact of the nasal mucosa (e.g., peppermint oil, N-tridecyl-beta-D-maltoside, and hexarelin). Chitosan, for example, has both mucoadhesive and penetration enhancing properties. Other agents that can be used to in formulations for intranasal delivery include liposomes (e.g., cationic liposomes and liposomes coated with polyethylene glycol (PEG), vasoconstrictors (e.g., phenylephrine), to limit absorption through the systemic pathway and increase absorption through the olfactory epithelium. Additional formulations and methods for intranasal administration are found in Illum, L., J Pharm Pharmacol, 56:3-17, 2004 and Illum, L., Eur J Pharm Sci 11:1-18, 2000, each of which is hereby incorporated by reference in its entirety.

Either of liquid and powder intranasal formulations may be used. Ketamine, for example, may be combined with a dispersing agent, or dispersant, and administered intranasally in an aerosol formulation optimized for intranasal administration.

Intranasal liquid aerosol formulations contain ketamine and a dispersing agent in a physiologically acceptable diluent. Aerosolized formulations are broken down into liquid or solid particles in order to ensure that the aerosolized dose actually reaches the mucous membranes of the nasal passages. The term “aerosol particle” is used to describe the liquid or solid particle suitable for intranasal administration, i.e., that will reach the mucous membranes. Other considerations, such as construction of the delivery device, additional components in the formulation, and particle characteristics are important. These aspects of intranasal administration of a drug are well known in the art, and manipulation of formulations, aerosolization means and construction of a delivery device require at most routine experimentation by one of ordinary skill in the art. In some embodiments, the mass median dynamic diameter will be 5 micrometers or less in order to ensure that the drug particles reach the lung alveoli (Wearley, L. L., 1991, 1991, Crit. Rev. in Ther. Drug Carrier Systems 8:333).

With regard to construction of the delivery device, any form of aerosolization known in the art, including but not limited to spray bottles, nebulization, atomization or pump aerosolization of a liquid formulation, and aerosolization of a dry powder formulation, can be used in the practice of the invention.

Intranasal aerosol formulations can also be prepared as a dry powder formulation comprising a finely divided powder form of ketamine and a dispersant. For example, the dry powder formulation can comprise a finely divided dry powder containing ketamine, a dispersing agent and also a bulking agent. Bulking agents useful in conjunction with the present formulation include such agents as lactose, sorbitol, sucrose, or mannitol, in amounts that facilitate the dispersal of the powder from the device.

Nasal formulations may be administered with the aid of a delivery device, e.g., an aerosol delivery. Any form of aerosolization known in the art, including but not limited to spray bottles, nebulization, atomization or pump aerosolization of a liquid formulation, and aerosolization of a dry powder formulation, can be used.

Nasal formulations may be administered, for example, using a plastic squeeze bottle with an aperture or opening dimensioned to aerosolize an aerosol formulation by forming a spray when squeezed. The opening is usually found in the top of the bottle, and the top is generally tapered to partially fit in the nasal passages for efficient administration of the aerosol formulation.

A useful device for intranasal administration is a small, hard bottle to which a metered dose sprayer is attached. In one embodiment, the metered dose is delivered by drawing the ketamine solution into a chamber of defined volume, which chamber has an aperture dimensioned to aerosolize and aerosol formulation by forming a spray when a liquid in the chamber is compressed. The chamber is compressed to administer the ketamine. In a specific embodiment, the chamber is a piston arrangement. Such devices are commercially available. In some embodiments, the metered dose is below the level associated with dysphoria or hallucination. In some embodiments, the metered dose is a dose of ketamine effective to treat epilepsy but which dose of ketamine is determined by a physician or medical care provider to be below a dose that causes anesthesia.

A preferred device for intranasal delivery of compositions and formulations is the OptiNose apparatus, which is commercially available from OptiNose US Inc. (Yardley, PA). In some embodiments, the OptiNose device is configured to deliver ketamine to the nasal olfactory epithelium. Delivery of ketamine to the nasal olfactory epithelium enables delivery of the ketamine through the nose-brain pathway directly into the cerebrospinal fluid.

Other devices useful for administering a dose intranasally are mucosal automation device that provide atomization of topical solution across the nasal and oropharyngeal mucous membranes that produce a typical particle size of 30 microns. An example of such a device is the LMA MAD Nasal™ device (LMA Company, San Diego, CA), which produces a typical particle size of 30 microns, has a system dead space of 0.09 mL, a tip diameter of about 3/16″ (4 mm), and an applicator length of about 1-¾″ (44 mm) can be used.

In another embodiment, intranasal drug delivery is achieved by taking a solubilized medication (liquid form) and dripping it into the nose a few drops at a time, allowing it to run down onto the nasal mucosa. This can be done using, e.g., a syringe.

In certain embodiments, the present disclosure provides liquid or powder aerosol formulations and dosage forms for intranasal administration (e.g., for use in treating subjects suffering from DRE). In general such dosage forms contain ketamine in a pharmaceutically acceptable diluent. Pharmaceutically acceptable diluents in such liquid aerosol formulations include but are not limited to sterile water, saline, buffered saline, dextrose solution, and the like. In a specific embodiment, a diluent that may be used in the present disclosure and/or in a pharmaceutical formulation of the present disclosure is phosphate buffered saline or a buffered saline solution generally between the pH 7.0-8.0 range, or water. The present disclosure contemplates the use of any suitable diluent known in the art for intranasal administration.

Formulations may also include other agents, ingredients, and/or components, e.g., that are useful for pH maintenance, solution stabilization, for the regulation of osmotic pressure solubility, drug stability, or enhanced absorption through nasal mucosa, including, but not limited to salts, such as sodium chloride, or potassium chloride, and carbohydrates, such as glucose, galactose or mannose, and the like.

Formulations for intranasal administration may include a “mucosal penetration enhancer,” i.e., a reagent that increases the rate or facility of transmucosal penetration of ketamine, such as but not limited to, a bile salt, fatty acid, surfactant or alcohol. Examples of penetration enhancers include sodium cholate, sodium dodecyl sulphate, sodium deoxycholate, taurodeoxycholate, sodium glycocholate, dimethylsulfoxide or ethanol.

Formulations disclosed herein, e.g., intranasal formulations, may include a dispersant. Preferably, a dispersant is pharmaceutically acceptable. Suitable dispersing agents are well known in the art, and include but are not limited to surfactants and the like. Such surfactants are generally used reduce surface induce aggregation caused by atomization of the solution forming a liquid aerosol. Examples of such surfactants include, but are not limited to, polyoxyethylene fatty acid esters and alcohols, and polyoxyethylene sorbitan fatty acid esters. Amounts of surfactants used will vary, being generally within the range or 0.001 and 4% by weight of the formulation. Suitable surfactants are well known in the art, and can be selected on the basis of desired properties, depending on the specific formulation.

In some embodiments, the ketamine is administered as an aerosol spray. In some embodiments, the ketamine is formulated as a dry powder. In some embodiments, the ketamine is administered by means of a device comprising a metered dose inhaler. In some embodiments, the ketamine or pharmaceutically acceptable salt thereof is administered by means of a device comprising a nasal spray inhaler containing an aerosol spray formulation of ketamine and a pharmaceutically acceptable dispersant, wherein the device is metered to disperse an amount of the aerosol formulation by forming a spray that contains a dose of ketamine effective to treat epilepsy but which dose of ketamine is determined by a physician or medical care provider to be below a dose that causes anesthesia. In some embodiments, the ketamine is formulated with a dispersant. In some embodiments, the ketamine is formulated with a mucosal penetration enhancer. Often, the aerosolization of a liquid or a dry powder formulation for inhalation into the lung will require a propellant. The propellant may be any propellant generally used in the art. Specific nonlimiting examples of such useful propellants are a chloroflourocarbon, a hydrofluorocarbon, a hydochlorofluorocarbon, or a hydrocarbon, including trifluoromethane, dichlorodiflouromethane, dichlorotetrafluoroethanol, and 1,1,1,2-tetraflouroethane, or combinations thereof. In some embodiments, the ketamine is formulated with a propellant.

Contemplated for use herein are oral solid dosage forms, which are described generally in Remington's Pharmaceutical Sciences, 18th Ed. 1990 (Mack Publishing Co. Easton PA 18042) at Chapter 89). Solid dosage forms include tablets, capsules, pills, troches or lozenges, cachets, pellets, powders, or granules. An exemplary lozenge formulation is described in Chong et al. Clin Drug Investig. 2009; 29(5):317-24. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for the therapeutic is given by Marshall, K. In: Modern Pharmaceutics Edited by G. S. Banker and C. T. Rhodes Chapter 10, 1979. In general, the formulation includes the therapeutic agent and inert ingredients which allow for protection against the stomach environment, and release of the biologically active material in the intestine.

Also contemplated for use herein are liquid dosage forms for oral administration, including pharmaceutically acceptable emulsions, solutions, suspensions, and syrups, which may contain other components including inert diluents; adjuvants, wetting agents, emulsifying and suspending agents; and sweetening, flavoring, coloring, and perfuming agents.

For oral formulations, the location of release may be the stomach, the small intestine (the duodenum, the jejunem, or the ileum), or the large intestine. One skilled in the art has available formulations which will not dissolve in the stomach, yet will release the material in the duodenum or elsewhere in the intestine, e.g., by the use of an enteric coating. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (i.e. powder), for liquid forms a soft gelatin shell may be used. The shell material of cachets could be thick starch or other edible paper. For pills, lozenges, molded tablets or tablet triturates, moist massing techniques can be used. The formulation of the material for capsule administration could also be as a powder, lightly compressed plugs, or even as tablets. These therapeutics could be prepared by compression.

One may dilute or increase the volume of the therapeutic agent with an inert material. These diluents could include carbohydrates, especially mannitol, lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic agent into a solid dosage form. Materials used as disintegrants include but are not limited to starch, including the commercial disintegrant based on starch, Explotab, Sodium starch glycolate, Amberlite, sodium carboxymethylcellulose, ultramylopectin, sodium alginate, gelatin, orange peel, acid carboxymethyl cellulose, natural sponge and bentonite may all be used. The disintegrants may also be insoluble cationic exchange resins. Powdered gums may be used as disintegrants and as binders, and can include powdered gums such as agar, Karaya or tragacanth. Alginic acid and its sodium salt are also useful as disintegrants. Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the peptide (or derivative).

An antifrictional agent may be included in the formulation to prevent sticking during the formulation process. Lubricants may be used as a layer between the peptide (or derivative) and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties drug during formulation and to aid rearrangement during compression might be added. The glidants may include starch, talc, pyrogenic silica and hydrated silicoaluminate.

To aid dissolution of the therapeutic agent into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the protein or derivative either alone or as a mixture in different ratios.

Controlled release oral formulations may used in practicing the present invention. The therapeutic agent could be incorporated into an inert matrix which permits release by either diffusion or leaching mechanisms, e.g., gums. Slowly degenerating matrices may also be incorporated into the formulation. Some enteric coatings also have a delayed release effect. Another form of a controlled release is by a method based on the Oros therapeutic system (Alza Corp.), i.e., the therapeutic agent is enclosed in a semipermeable membrane which allows water to enter and push agent out through a single small opening due to osmotic effects. In some embodiments, the ketamine is formulated to maintain the plasma level of the ketamine in the subject at 10% or greater (e.g., 15% or greater, 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, or 95% or greater) of the peak ketamine plasma level (i.e., the Cₘₐₓ of ketamine) for at least 6 hours (e.g., at least 8 hours, at least 12 hours, at least 16 hours, at least 20 hours, at least 24 hours, at least 36 hours, or at least 48 hours) after administration of the ketamine. It is understood that the peak ketamine plasma level (i.e., the Cₘₐₓ of ketamine) is the highest plasma concentration of the ketamine observed in the subject after administration of the ketamine. In some embodiments peak ketamine plasma level (i.e., the Cₘₐₓ of ketamine) is from about 0.1 ng/mL to about 2000 ng/mL. For example, from about 0.1 ng/mL to about 2000 ng/mL, from about 0.1 ng/mL to about 1500 ng/mL, from about 0.1 ng/mL to about 1200 ng/mL, from about 0.1 ng/mL to about 1000 ng/mL, from about 0.1 ng/mL to about 800 ng/mL, from about 0.1 ng/mL to about 600 ng/mL, from about 0.1 ng/mL to about 500 ng/mL, from about 0.1 ng/mL to about 400 ng/mL, from about 0.1 ng/mL to about 300 ng/mL, from about 0.1 ng/mL to about 200 ng/mL, from about 0.1 ng/mL to about 100 ng/mL, from about 0.1 ng/mL to about 70 ng/mL, from about 0.1 ng/mL to about 50 ng/mL, from about 0.1 ng/mL to about 40 ng/mL, from about 0.1 ng/mL to about 30 ng/mL, from about 0.1 ng/mL to about 20 ng/mL, from about 0.1 ng/mL to about 10 ng/mL, from about 0.1 ng/mL to about 5 ng/mL, from about 20 ng/mL to about 50 ng/mL, from about 25 ng/mL to about 35 ng/mL, from about 28 ng/mL to about 32 ng/mL, from about 200 ng/mL to about 800 ng/mL, from about 300 ng/mL to about 700 ng/mL, from about 400 ng/mL to about 600 ng/mL, from about 450 ng/mL to about 550 ng/mL, from about 480 ng/mL to about 520 ng/mL, about 10 ng/mL, about 20 ng/mL, about 30 ng/mL, about 40 ng/mL, about 50 ng/mL, about 100 ng/mL, about 150 ng/mL, about 200 ng/mL, about 300 ng/mL, about 400 ng/mL, about 500 ng/mL, about 600 ng/mL, or about 700 ng/mL.

In some embodiments, the tₘₐₓ is from about 1 minute to about 10 hours. For example, from about 1 minute to about 5 minutes, from about 1 minute to about 10 minutes, from about 1 minute to about 15 minutes, from about 1 minute to about 30 minutes, from about 1 minute to about 1 hour, from about 1 minute to about 1.5 hours, from about 1 minute to about 2 hours, from about 1 minute to about 3 hours, from about 1 minute to about 5 hours, from about 1 minute to about 7 hours, from about 30 minutes to about 1.5 hours, from about 45 minutes to about 1.25 hours, from about 50 minutes to about 70 minutes, from about 1 hour to about 2 hours, from about 1 hour to about 3 hours, from about 1 hour to about 4 hours, from about 1 hour to about 5 hours, about 15 minutes, about 45 minutes, about 50 minutes, about 55 minutes, about 1 hour, about 1.1 hours, about 1.5 hours, about 2 hours, or about 3 hours.

Other coatings may be used for the formulation. These include a variety of sugars which could be applied in a coating pan. The therapeutic agent could also be given in a film coated tablet and the materials used in this instance are divided into 2 groups. The first are the nonenteric materials and include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid. A mix of materials might be used to provide the optimum film coating. Film coating may be carried out in a pan coater or in a fluidized bed or by compression coating.

In another alternative embodiment, administration comprises transdermal administration. Transdermal administration includes passive or active transdermal or transcutaneous modalities, including, for example, patches and iontophoresis devices, as well as topical application of pastes, salves, or ointments.

Those of skill in the art are well aware of general technologies for transdermal drug delivery or administration of a therapeutic agent to the skin. Transdermal drug delivery offers controlled release of a drug to the patient and transdermal patches are user-friendly, convenient, painless, and offer multi-day dosing which usually results in improved patient compliance. The methods of the invention for treating DRE patients with a transdermal administration of ketamine can include administering ketamine to skin of the face, head or body. Such a ketamine composition can be administered to the skin of the face, scalp, temporal region, arms, stomach, thighs, back, neck and the like. Suitable skin of the face includes skin of the chin, the upper lip, the lower lip, the forehead, the nose, the cheek, the skin around the eyes, the upper eyelid, the lower eyelid or combinations thereof. Suitable skin of the scalp includes the front of the scalp, the scalp over the temporal region, the lateral part of the scalp, or combinations thereof. Suitable skin of the temporal region includes the temple and the scalp over the temporal region and combinations thereof. The ketamine may be formulated into a bioadhesive patch or a bioadhesive strip with an occlusive covering. Alternatively, the transdermal ketamine composition for administration to the skin can be applied as a topical ointment, a topical gel, a lotion, a cream, a solution, a spray, a paint, a film, a foil, a cosmetic, to be applied to the skin in a layer with or without an occlusive dressing.

Intradermal administration of ketamine-containing compositions also is contemplated. Intradermal administration of a therapeutic agent is defined as within or between the layers of skin. In contrast, subcutaneous administration is defined as beneath the initial layer of skin and intravenous is a systemic administration into the bloodstream. Administration of therapeutic agents by intradermal, intravenous or subcutaneous injection is a common means of drug delivery and readily performed by one skilled in the art.

The compositions and formulation described herein may be administered by a health professional or by a patient. Patient self-administration of ketamine to treat DRE is expressly contemplated. Intranasal administration and administration via transdermal patch are particularly suited to patient self-administration.

Formulations for use in the methods described herein can include other therapeutically or pharmacologically active ingredients in addition to ketamine.

Effective amounts of ketamine in compositions including pharmaceutical formulations, include doses that partially or completely achieve the desired therapeutic, prophylactic, and/or biological effect. In a specific embodiment, an effective amount of ketamine administered to a subject with DRE is effective for treating one or more signs or symptoms of DRE. The actual amount effective for a particular application depends on the condition being treated and the route of administration.

In certain aspect, the present disclosure provides for administration of a therapeutically effective dose of ketamine, i.e., a dose effective to treat DRE. Specific dosages may be adjusted depending on conditions of disease, i.e., the severity of DRE, the age, body weight, general health conditions, sex, and diet of the subject, dose intervals, administration routes, excretion rate, and combinations of drugs. Any of the dosage forms described herein containing effective amounts of ketamine, either alone or in combination with one or more active agents, are well within the bounds of routine experimentation and therefore, well within the scope of the instant invention.

An initial dose may be larger, followed by smaller maintenance doses. The dose may be administered as infrequently as weekly or biweekly, or fractionated into smaller doses and administered daily, several times daily, semi-weekly, bi-weekly, quarterly, etc., to maintain an effective dosage level. Preliminary doses can be determined according to animal tests, and the scaling of dosages for human administration can be performed according to art-accepted practices. In certain embodiments, a subject may be administered 1 dose, 2 doses, 3 doses, 4 doses, 5 doses, 6 doses or more of a ketamine-containing composition described herein. However, other ranges are possible, depending on the subject's response to the treatment. Moreover, an initial dose may be the same as, or lower or higher than subsequently administered doses of ketamine.

The number and frequency of doses may be determined based on the subject's response to administration of the composition, e.g., if one or more of the patient's symptoms improve and/or if the subject tolerates administration of the composition without adverse reaction; in some subjects, a single dose is sufficient, other subjects may receive a daily, several times a day, every other day, several times per week, weekly, biweekly, semi-weekly, or monthly administration of a composition containing ketamine as described herein. The duration and frequency of treatment will depend upon the subject's response to treatment, i.e., if the subject's condition and/or one more symptoms of DRE improves. In one example of a dosing regimen, an initial dose of ketamine is used to treat DRE, followed by titration of to a lower dose of ketamine to maintain treatment of the DRE. Such a regimen may be particularly useful, for example, to use a high dose of ketamine to treat acute symptoms of DRE, followed by titrating to a lower dose of ketamine, to treat chronic symptoms of DRE.

In some embodiments, a dose of ketamine to treat DRE is approximately 0.001 to approximately 2 mg/kg body, 0.01 to approximately 1 mg/kg of body weight, or approximately 0.05 to approximately 0.7 mg/kg of body weight. A subject (e.g., patient) suffering from DRE may be administered (including self administration) a dose of ketamine of, for example, about 0.01 mg per kg of body weight (mg/kg), about 0.05 mg/kg, 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2 mg/kg, or about 3 mg/kg.

In some embodiments, the total dose of ketamine for, e.g., intranasal, transdermal, intravenous, intradermal, or subcutaneous administration (e.g., intravenous or intranasal administration) ranges from about 1 mg to about 250 mg (e.g., about 1 mg to about 10 mg, about 1 mg to about 40 mg, about 1 mg to about 100 mg, about 1 mg to about 175 mg, about 20 mg to about 40 mg, about 20 mg to about 100 mg, about 20 mg to about 175 mg, about 50 mg to about 100 mg, about 100 mg to about 150 mg, about 150 mg to about 200 mg, or about 200 mg to about 250 mg, about 1 mg, about 2 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, and about 250 mg).

In some embodiments, an intranasal, transdermal, intravenous, intradermal, or subcutaneous (e.g., intravenous or intranasal) dose of ketamine for a subject of 80 kg body weight is equal to or greater than about 40 mg, for example, about 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, or 250 mg. In certain embodiments, intranasal administration of 8-32 mg of ketamine, corresponding to 0.13 to 0.53 mg/kg of body weight is contemplated. In some embodiments, the effective dose is titrated under the supervision of a physician or medical care provider, so that the optimum dose for the particular application is accurately determined. A dose suited to each individual patient is thus provided. Once the dosage range is established, a further advantage is that the patient can administer ketamine on an as-needed, dose-to-effect basis. Thus, the frequency of administration is under control of the patient. However, the relatively low dose with each administration will reduce the possibilities for abuse.

In some embodiments, a dose of esketamine to treat DRE is approximately 0.001 to approximately 2 mg/kg body, 0.01 to approximately 1 mg/kg of body weight, or approximately 0.05 to approximately 0.7 mg/kg of body weight. A subject (e.g., patient) suffering from DRE may be administered (including self-administration) a dose of esketamine of, for example, about 0.01 mg per kg of body weight (mg/kg), about 0.05 mg/kg, 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2 mg/kg, or about 3 mg/kg.

In another embodiment, the total dose of esketamine per intranasal administration ranges from about 1 to about 250 mg. By way of non-limiting example, esketamine doses of 1 mg, 2 mg, 4 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, and 250 mg are specifically contemplated.

In another embodiment, the total dose of ketamine per intranasal administration ranges from about 10 mg to about 300 mg, about 10 mg to about 250 mg, about 10 to about 200 mg, about 15 to about 175 mg, about 20 to about 175 mg, about 25 to about 150 mg, about 25 to about 125 mg, about 25 to about 100 mg, about 50 to about 100 mg, about 50 mg to about 75 mg, about 75 mg to about 100 mg, or about 75 mg to about 200 mg.

In another embodiment, the total dose of esketamine per intranasal administration ranges from about 10 mg to about 300 mg, about 10 mg to about 250 mg, about 10 to about 200 mg, about 15 to about 175 mg, about 20 to about 175 mg, about 25 to about 150 mg, about 25 to about 125 mg, about 25 to about 100 mg, about 50 to about 100 mg, about 50 mg to about 75 mg, about 75 mg to about 100 mg, or about 75 mg to about 200 mg.

In a certain embodiments, an intranasal or intravenous dose of esketamine for a subject of about 70-80 kg body weight is equal to or greater than about 40 mg, for example, about 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, or 250 mg.

In certain embodiments, intranasal administration of 8-32 mg of esketamine, corresponding to 0.13 to 0.53 mg/kg of body weight is contemplated. In another embodiment, intranasal administration of a total dose of between about 50-75 mg of esketamine, corresponding to between about 0.83 to 1.25 mg/kg of body weight is contemplated. In another embodiment, intranasal administration of a total dose of between about 50-75 mg of esketamine, corresponding to between about 0.74 to 1.1 mg/kg of body weight is contemplated.

In certain embodiments, intranasal administration of 8-32 mg of ketamine, corresponding to 0.13 to 0.53 mg/kg of body weight is contemplated. In another embodiment, intranasal administration of a total dose of between about 50-75 mg of ketamine, corresponding to between about 0.83 to 1.25 mg/kg of body weight is contemplated. In another embodiment, intranasal administration of a total dose of between about 50-75 mg of ketamine, corresponding to between about 0.74 to 1.1 mg/kg of body weight is contemplated.

Preferably, the effective dose of ketamine is titrated under the supervision of a physician or medical care provider, so that the optimum dose for the particular application is accurately determined. Thus, the present disclosure provides a dose suited to each individual subject (e.g., patient).

Once a dosage range is established, an advantage of compositions for intranasal administration of ketamine and methods of treatment via intranasal administration is that the patient can administer (e.g., self-administer) ketamine on an as-needed, dose-to-effect basis. Thus, the frequency of administration is under control of the subject. Yet another particular advantage is that intranasal administration of ketamine is non-invasive, and facilitates ketamine's crossing of the blood-brain barrier.

The mild adverse effects of ketamine, e.g., dysphoria and/or hallucinations, sometimes called “ketamine dreams,” can occur upon administration of a dose of greater than 50 mg of ketamine, and usually require doses greater than 100 mg of ketamine of total dose intranasally. When administering ketamine to treat DRE, it is preferable to administer a dose that is effective in treating DRE, but is below the level that results in such side effects. It is possible, however, that higher doses of ketamine may be administered, particularly in response to an acute episode of DRE.

Methods for treating a human patient with DRE are directed to using ketamine to reduce or eliminate at least one symptom of DRE in the patient. Ketamine may be administered as a racemic mixture of (S)-ketamine and (R)-ketamine, or as enantiomerically enriched for a ketamine enantiomer. A composition may be enriched to the extent that it is, for example, 90%, 95%, 99%, 99.9 or 99.99% of either of the (S)-ketamine and (R)-ketamine enantiomer.

In a specific embodiment, a method for treating a human patient with DRE is directed to using esketamine to reduce or eliminate at least one symptom of DRE in the patient.

In certain embodiments, a composition comprising ketamine is administered intranasally or intravenously to a patient suffering from DRE.

In other embodiments, the present disclosure also contemplates the prophylactic use of the ketamine-containing compositions and formulations disclosed herein. For example, in certain embodiments, presently provided are methods for inhibiting development of DRE in a human patient which comprises administering to a subject in need of such inhibiting a composition comprising a therapeutically effective amount of ketamine for inhibiting the development of DRE and/or one the development of one or more DRE-like symptoms, wherein the therapeutically effective amount is a dosage range of about dose of between about 0.1 mg/kg per day to about 3.0 mg/kg/dayIn specific embodiments, the symptoms of DRE are alleviated within 2 hours of administration of the ketamine. As disclosed herein, symptoms of DRE may be alleviated concomitant with administration of ketamine.

IV administration of ketamine can be on an as needed basis, e.g., when symptoms of DRE appear. For IV administration, ketamine (e.g., doses of at least 0.5 mg/kg) may be administered over a period of 40 minutes. IV administration may be continued for up to 1 week, or longer. IV administration of ketamine may also be effected at least twice, at least three times, at least four times, at least five time, at least six times, at least seven times per week, and may be continued over a period of two, three, four, five, six, seven, eight, nine or 10 weeks, or more. No serious adverse events caused by IV administration of ketamine have been observed. Any side effects observed are typically mild, e.g., euphoria, elevated BP, increased libido, perceptual disturbances, and furthermore these effects typically abate within 80 minutes post-infusion.

Administration of intranasal ketamine can be on an as needed basis, e.g., when symptoms of DRE appear. In specific embodiments, the ketamine is administered at least two, at least three, at least four, at least five, at least six, at least seven, at least eight, at least nine times in fourteen days. In other embodiments, the ketamine is administered at least two, at least three, at least four, at least five, at least six, at least seven, at least eight, at least nine times in twenty-one days. In other embodiments, the intranasal ketamine is administered at least once a day, at least twice a day, at least three times per day, or more. In other embodiments, the intranasal ketamine is administered at least once a week, at least twice a week, at least three times per week, or more frequently. In another embodiment, the intranasal ketamine is administered at least twice per months, or at least once per months. Treatment can continue as long as needed.

In some embodiments, a second agent is used in combination with ketamine to treat DRE, or following an initial treatment phase of DRE with ketamine, wherein the second agent boosts the positive effect or ketamine in treatment of DRE or sustains the positive effect of ketamine in treatment of DRE.

In some embodiments, intravenous, oral, buccal, sublingual, pulmonary, and transdermal administration of ketamine are contemplated. In one alternative embodiment, the invention thus provides a method of treating a human patient for DRE, comprising intravenously administering a composition comprising ketamine to the patient at a dosage sufficient to reduce or eliminate the symptoms of the DRE. In another alternative embodiment, the invention thus provides a method of treating a human patient for DRE, comprising transdermally administering a composition comprising ketamine to the patient at a dosage sufficient to reduce or eliminate the symptoms of the DRE. In another alternative embodiment, the invention thus provides a method of treating a human patient for DRE, comprising orally (e.g., liquid or solid (e.g., lozenge) dosage form) administering a composition comprising ketamine to the patient at a dosage sufficient to reduce or eliminate the symptoms of the DRE. In more specific embodiments, the ketamine is in a pharmaceutically acceptable carrier and is administered at a dose of between about 0.1 mg/kg per day to about 3.0 mg/kg/day.

The methods of the invention may be achieved through a method that comprises intravenous, oral, or transdermal administration of multiple doses of the ketamine. Administration intravenous, oral, or transdermal administration ketamine can be on an as needed basis, e.g., when symptoms of DRE appear. In specific embodiments, the ketamine is administered at least two, at least three, at least four, at least five, at least six, at least seven, at least eight, at least nine times in fourteen days. In other embodiments, the ketamine is administered at least two, at least three, at least four, at least five, at least six, at least seven, at least eight, at least nine times in six week. In other embodiments, the ketamine is administered at least once a day, at least twice a day, at least three times per day, or more. In other embodiments, the ketamine is administered at least once a week, at least twice a week, at least three times per week, or more frequently. In another embodiment, the ketamine is administered at least twice per months, or at least once per months. Treatment can continue as long as needed.

### Example

This is a pilot study to study to assess the efficacy of a sub anesthetic dose of IV Ketamine in the treatment Drug Resistant Epilepsy in outpatient setting.

### Study Description:

Ketamine is a medication that came into clinical practice in the 1960's.
Ketamine is used as an anesthetic and to provide pain relief. Recently, Ketamine was approved to treat drug resistant depression using subanesthetic doses. In the hospital setting, intravenous anesthetic dosages are used to treat unrelenting seizures known as status epilepticus in comatose patients. Ketamine in subanesthetic doses has not been tried as a treatment for medication resistant seizures in the outpatient setting. This study would like to examine the effectiveness of subanesthetic ketamine in outpatients who suffer from drug resistant epilepsy.

### Objectives:

Primary Objective: To quantify the seizure frequency in medication resistant patients treated with ketamine. Our hypothesis is that ketamine would reduce seizure frequency in this population
Secondary Objectives: Note the presence or absence of depression/anxiety in this patient population and quantify if Ketamine has an anti-depressant/anxiolytic effect.

### Endpoints:

Primary Endpoint: Adjunctive ketamine will significantly reduce the seizure frequency per 28 days. During the 2 week period of active treatment, a 50% seizure reduction is expected. During the 28 days post-infusion a continued 50% seizure reduction is expected. At 3 months a return to pre-ketamine infusion seizure frequency is expected.
Secondary Endpoints: Mood assessment. Although depression and/or anxiety is not an inclusion criteria a NDDI-E, QOLIE-10, GAD 7, ADAMS will be performed pre and post treatment. An improvement in mood assessments is expected.

### Study Population: Drug Resistant Epilepsy Patients Phase or Stage:

Phase IIa

### Description of Sites/

Epilepsy Outpatient Clinic

### Facilities Enrolling Participants:

### Study Duration:

2 years

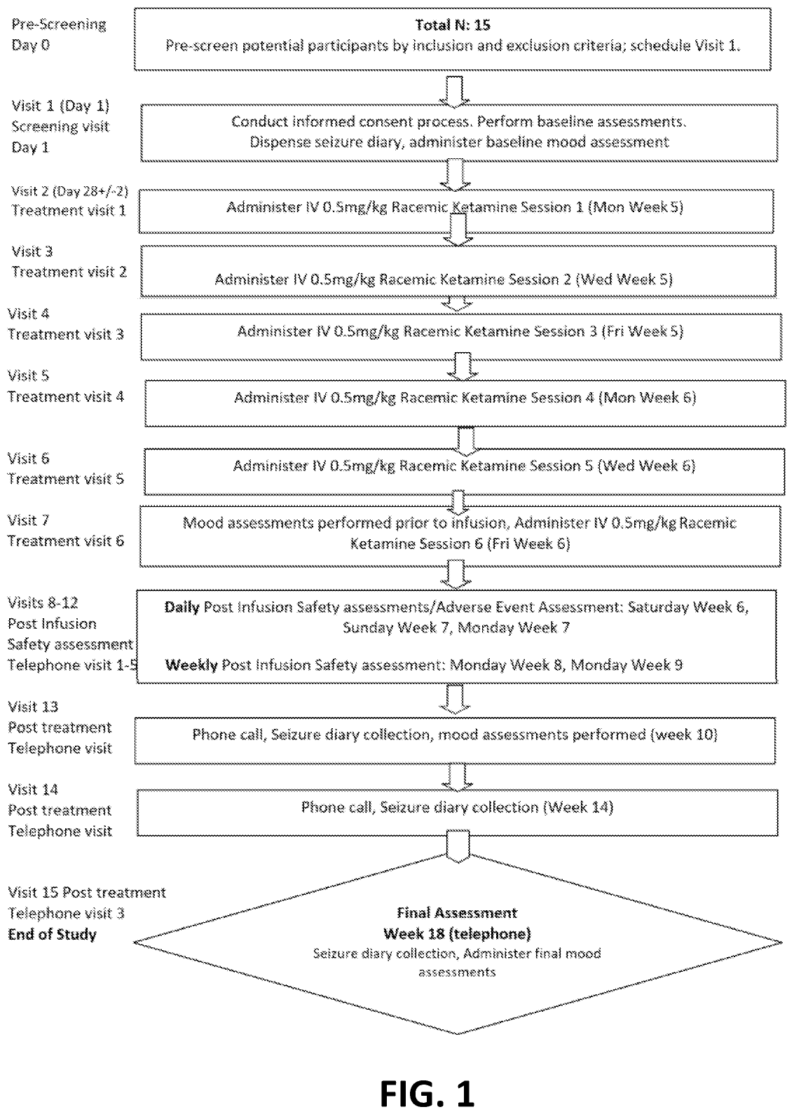

FIG. 1 depicts a flowchart depicting assessments and treatments for the patients in the study. Table 1 depicts the schedule of activities for the study.

| | Period | | | | | | | | | | | |
|---|---|---|---|---|---|---|---|---|---|---|---|---|
| | | | | | | | | Post Infusion | | | | |
| | Screening | Treatment Visits | | | | | | Safety Assessment | | Post Treatment Visits | | |
| | Study Week(s) | | | | | | | | | | | |
| | | | | | | | | 3 Day | Weekly | | | |
| | | Wk 5 | Wk 5 | Wk 5 | Wk 6 | Wk 6 | Wk 6 | (Daily) | Wk 8 | Week | Week | Week |
| | Week 1 | Mon | Wed | Fri | Mon | Wed | Fri | Sat-Mon | Wk 9 | 10 | 14 | 18 |
| Visit Number | 1 | 2 | 3 | 4 | 5 | 6 | 7 | 8, 9, 10 | 11, 12 | 13 | 14 | 15 |
| Informed Consent | X | | | | | | | | | | | |
| Inclusion/Exclusion | X | | | | | | | | | | | |
| Medical History | X | | | | | | | | | | | |
| Seizure Type | X | | | | | | | | | | | |
| and Frequency | | | | | | | | | | | | |
| Concomitant and | X | | | | | | | | | | | |
| Seizure | | | | | | | | | | | | |
| Medications | | | | | | | | | | | | |
| Dispense | X | | | | | | | | | | | |
| Subject Diary | (28 days) | | | | | | | | | | | |
| Vital Signs | | X | X | X | X | X | X | | | | | |
| Weight (kg) | | X | X | X | X | X | X | | | | | |
| Spot | | X | | | | | | | | | | |
| Pregnancy | | | | | | | | | | | | |
| Test (Urine) | | | | | | | | | | | | |
| Administer IV | | X | X | X | X | X | X | | | | | |
| 0.5 mg/kg | | | | | | | | | | | | |
| Racemic Ketamine | | | | | | | | | | | | |
| NDDI-E | | X | | | | | X | | | X | X | X |
| QOLIE-10 | | X | | | | | X | | | X | X | X |
| GAD-7 | | X | | | | | X | | | X | X | X |
| ADAMS | | X | | | | | X | | | X | X | X |
| Subject Diary | | X | X | X | X | X | X | | | X | X | X |
| Return Review | | | | | | | | | | | | |
| Adverse Events | | X | X | X | X | X | X | X | X | X | X | X |

Hemodynamic instability during infusion poses a potential but very low risk to the patient. Patients with a history of cardiovascular disease will be excluded from the study.

Adverse psychiatric effects during infusion may occur. These effects are reversible and will not cause permanent irreversible injury to the patient.

A physician and CRU staff will be present during the infusion and will continue to monitor the patient for 2 hours post infusion. Patients will be allowed to leave if deemed stable after assessment of post-infusion vitals.

The end of the study is defined as completion of the 3-month follow-up assessment shown in the above Schedule of Activities (SoA).

This is a single site open label pilot study to investigate the efficacy and clinical usability of a sub anesthetic dose (0.5 mg/kg) of IV Racemic Ketamine in the adult patients with Drug Resistant Epilepsy (DRE). 15 subjects will be enrolled in the study (this accounts for screen failures, only 10 subjects will qualify for the treatment phase i.e. enrollment will end when 10 subjects qualify to enter treatment phase).

The study consists of 3 phases: Screening—prior to entering the CRU; Treatment in the clinical research unit (CRU) where IV ketamine will be administered; Post-Treatment Safety Follow up. Subjects or the subject's legally authorized representative (LAR) will provide appropriately-obtained informed consent prior to completing any study-related procedures.

### Inclusion Criteria

In order to be eligible to participate in this study, an individual must meet all of the following criteria:
1. Provision of signed and dated informed consent form
2. Adults (18 years or older)
3. Cognitively impaired adults are not excluded (i.e. will be included in the study)
4. Established diagnosis of Drug Resistant Epilepsy (DRE) i.e. failed two or more appropriately chosen anti-seizure medications (ASMs)
5. EEG consistent with focal or generalized epilepsy
6. Patients must have >4 focal aware, focal impaired aware, focal to bilateral tonic clonic or generalized tonic clonic seizures per month.
7. Patients can be on >/=1 anti-seizure medication (ASM) at the time of enrollment on stable doses 12 weeks prior to initiation
8. Patients on Epilepsy devices: Vagal nerve stimulator (VNS), Deep brain stimulator (DBS) or Responsive Nerve Stimulator (RNS) must have remained stable for at least 4 weeks before the screening visit. Adjustment of devices is not allowed during the study.

### Exclusion Criteria

An individual who meets any of the following criteria will be excluded from participation in this study:
1. Patients <18 years of age
2. Pregnant women
3. Women that are breast feeding
4. Patients who had >21 days of seizure freedom in the last year.
5. Patients with a history of status epilepticus within 3 months of screening
6. Patients with a history of alcoholism of drug misuse within the last 2 years
7. Unstable medical illness
8. Serious or imminent suicidal or homicidal risk
9. Patients with cardiovascular disease
10. Patients with schizophrenia
11. Patients with history of aneurysm or aortic dissection, arteriovenous malformation and intracerebral hemorrhage
12. Patients that are immobile i.e. wheel chair bound, bed ridden individuals
13. Patients on psychostimulants (amphetamines, methylphenidate etc.) and Monoamine oxidase inhibitors (selegiline, isocarboxazid, phenelzine etc.)

### Strategies for Recruitment and Retention

The epilepsy providers at Mount Sinai see well over 3000 epilepsy people per year. Approximately 50% are drug resistant. A smaller percentage of those people will have the seizure frequency necessary to be included in the study. 15 patients will be recruited directly from Epilepsy outpatient clinic. Potential subjects will be approached by their treating epilepsy provider, who will briefly introduce the research and gauge interest. Potential subjects will then be asked for their permission to be contacted by a member of the research team.

Participants will not be compensated or provided any incentives (e.g. vouchers, gift cards,) for study participation.

Cognitively impaired adults will be enrolled in thus study. Assent will be obtained from any subject unless the capability of the subject is so limited that he/she cannot reasonably be consulted. The study will be explained in lay language and oral assent obtained. If an individual does not give assent, that person will not be included in the study. The assent checkbox in the consent form will be used to document assent.

### Screening Visit:

Informed consent will be obtained from either subject or subject's LAR by research staff, after which inclusion and exclusion criteria will be reviewed with patient. Seizure diary will be dispensed to subject and/or LAR. If patient consents to the study a history and physical examination, routine hematological tests (CBC, CMP at least within the last 3 months) and EKG will be performed. This is to establish medical illnesses. Seizure diary will be prospectively filled out in the 4 weeks prior to the 1st infusion. Baseline mood assessment will be performed at this screening visit.

### Treatment Phase:

Patients deemed eligible will proceed to the treatment phase. This phase will consist of 6 study visits (3 visits/week for 2 weeks).
Treatment Visit 1: Monday Week 5 (baseline seizures diary collected)
Treatment Visit 2: Wednesday Week 5
Treatment Visit 3: Friday Week 5
Treatment Visit 4: Monday Week 6
Treatment Visit 5: Wednesday Week 6
Treatment Visit 6: Friday Week 6 (Mood assessments performed prior to infusion)

Each visit is expected to last a duration of 3 hours. Treatment will take place at the Clinical Research Unit (CRU) at Mount Sinai Hospital (MSH).

Vitals (blood pressure, pulse, respiratory rate, SP02) will be taken pre-infusion and monitored throughout stay at the CRU. Weight will be measured and dose of study medication to be administered calculated. Pre-treatment mood assessments will be administered to the patients (NDDI-E, QOLIE-10, GAD 7, ADAMS). The mood assessments will be re-administered at treatment visit 6 prior to the infusion and again at 1 month and 3 months post-infusion visits. A point of care urine pregnancy test will be conducted on women of childbearing at Treatment Visit 1 on entry into the CRU, if positive, subject will be withdrawn from study.

Patients will receive 0.5 mg/kg Racemic ketamine IV over 40 min three times a week (M, W, F) for 2 consecutive weeks. Patients will be asked to fast overnight ahead of treatment visit (NPO after midnight until 1 hour post end of ketamine infusion). Patients are allowed to take their morning meds with a sip of water.

The infusions will take place in the CRU at MSH.

A physician and CRU staff will be present during the infusion and will continue to monitor the patient for 2 hours post infusion. Patients will be allowed to leave if deemed stable after assessment of post-infusion vitals.

While emergent intubation is unlikely, the protocol for respiratory depression or any emergency will be to call for a rapid response (or call 911). A physician will be with the patient at all times in the CRU to monitor for safety.

### Post-Treatment Phase

This phase will consist of 5 post infusion safety assessments and 3 post-treatment assessments.

Follow up safety assessments will be conducted virtually to monitor for adverse events. These will be conducted daily for the first three days after the last dose of ketamine and then weekly for a total of two weeks.

Follow up phone calls will be placed to patients at 1, 2 and 3 months post-infusion. Post-treatment mood assessments (NDDIE-E, QOLIE-10. GAD 7, ADAMS) will be administered to subjects at month 1 and 3 post-infusion. Seizure diary will be reviewed and adverse events will be assessed at each visit.
Post-Infusion Safety Assessment 1: Saturday Week 6 (Adverse Event Assessment)
Post-Infusion Safety Assessment 2: Sunday Week 7 (Adverse Event Assessment)
Post-Infusion Safety Assessment 3: Monday Week 7 (Adverse Event Assessment)
Post-Infusion Safety Assessment 4: Monday Week 8 (Adverse Event Assessment)
Post-Infusion Safety Assessment 5: Monday Week 9 (Adverse Event Assessment)
Post-Treatment Assessment 1: phone call week 10 (Seizure diary collection, mood assessments performed)
Post-Treatment Assessment 2: phone call week 14 (Seizure diary)
Post-Treatment Assessment 3: phone call week 18 (Seizure diary collection, mood assessments performed)

A participant is considered to have completed the study if he or she has completed the baseline assessment, 6 intervention sessions, and the 1, 2, and 3-month follow-up assessments.

Vitals (blood pressure, pulse, respiratory rate, SP02) will be taken pre-infusion and monitored throughout stay at the CRU. Weight will be measured and dose of study medication to be administered calculated. Patients will receive 0.5 mg/kg Racemic ketamine IV over 40 min three times a week (M, W, F) for 2 consecutive weeks.

A physician and CRU staff will be present during the infusion and will continue to monitor the patient for 2 hours post infusion. Patients will be allowed to leave if deemed stable after assessment of post-infusion vitals.

Patients on ASM/AED must be on stable doses 12 weeks prior to screening visit. Patients on Epilepsy devices: Vagal nerve stimulator (VNS), Deep brain stimulator (DBS) or Responsive Nerve Stimulator (RNS) must have remained stable for at least 4 weeks before the screening visit. Adjustment of devices is not allowed during the study.

The investigator may withdraw the subject from the study at any time for safety or administrative reasons.

### Reasons for Withdrawal are as the Following:

Allergic or adverse reaction (serious adverse event) during infusion.
Hemodynamic instability during infusion.

### Procedures for Orderly Termination

Subjects may withdraw from the research at any time for any reason. Subjects are asked to do so verbally, or in writing if they prefer. Subjects withdrawn by the investigator will be monitored and stabilized in the CRU. Adverse events resulting in subject early termination will be followed to the satisfactory resolution and determination of outcome, as ascertained by the Investigator. The reason for withdrawal and any adverse events and assessments will be documented.

If the patient is able to undergo partial but not all of the infusion protocol their data will be retained by the study team and may be used in the analysis.

Participants are free to withdraw from participation in the study at any time upon request.

A participant will be considered lost to follow-up if he or she fails to return for scheduled treatment visits and study staff are unable to contact the participant after at least 3 attempts.

The following actions must be taken if a participant fails to return to the clinic for a required study visit:
The site will attempt to contact the participant, reschedule the missed visit within 2 days, counsel the participant on the importance of maintaining the assigned visit schedule and ascertain if the participant wishes to and/or should continue in the study
Before a participant is deemed lost to follow-up, the investigator or designee will make every effort to regain contact with the participant (where possible, 3 telephone calls).
Should the participant continue to be unreachable, he or she will be considered to have withdrawn from the study with a primary reason of lost to follow-up]
Physical examination-based assessments: weight, blood pressure, heart rate, SP02.
Seizure diary log (28 days prior to 1st treatment visit—3 months post 6th treatment visit)
Mood assessments (NDDI-E, QOLIE-10, GAD-7, ADAMS) baseline, pre and post infusion

Vitals (blood pressure, pulse, respiratory rate, SP02) will be taken pre-infusion and monitored throughout infusion of IV Racemic Ketamine, and 2 hours post-infusion.

Follow up safety assessments will be conducted virtually to monitor for adverse events.

These will be conducted daily for the first three days after the last dose of ketamine and then weekly for a total of two weeks. Symptoms assessed will include but not limited to: craving, dysphoria, shaking, sweating, palpitations, tiredness, low appetite, low mood, chills, autonomic arousal, lacrimation, restlessness, anxiety, nightmares, paranoia, delusions, and hallucinations, agitation, confusion, loss of motor skills, rage, nausea, decreased respiratory and cardiac functions, insomnia, cognitive impairment, and tremors.

While emergent intubation is unlikely, the protocol for respiratory depression or any emergency will be to call for a rapid response (or call 911). A physician will be with the patient at all times in the CRU to monitor for safety.

Adverse events resulting in subject early termination will be followed to the satisfactory resolution and determination of outcome, as ascertained by the Investigator. The reason for withdrawal and any adverse events and assessments will be documented.

### Abuse-Related Adverse Events

Abuse: Sporadic or persistent intentional excessive use of study drug accompanied by harmful physical or psychological effects.

Abuse Related AEs to be monitored include:
Euphoric mood; Elevated mood; Feeling abnormal; Feeling drunk; Feeling of relaxation;
Dizziness; Thinking abnormal; Hallucination; Inappropriate affect
Impaired attention, cognition, and mood; Somnolence, Mood disorders and disturbances
Psychosis; Aggression; Confusion and disorientation
Drug accountability discrepancies: diversion of study medication

All AEs associated with abuse will be captured as an AE and must be reported in an expedited manner.

All statistical analyses will be performed by the investigator or designee after the study is completed. Statistical analyses will be performed using SAS software or other validated statistical software as required.

As part of administering the informed consent document, the investigator/research staff will explain to each subject and/or guardian/legally authorized representative the nature of the study, its purpose, the procedures involved, the expected duration, the potential risks and benefits involved, any potential discomfort, potential alternative procedure(s) or course(s) of treatment available to the subject, and the extent of maintaining confidentiality of the subject's records. Each subject will be informed that participation in the study is voluntary, that he/she may withdraw from the study at any time, and that withdrawal of consent will not affect his/her subsequent medical treatment or relationship with the treating physician.

This informed consent should be given by means of a standard written statement, written in nontechnical language. The subject and/or the subject's legally acceptable representative should understand the statement before signing and dating it and will be given a copy of the signed document. If a subject is unable to read or if a legally acceptable representative is unable to read, an impartial witness should be present during the entire informed consent discussion. After the ICF and any other written information to be provided to subjects is read and explained to the subject or the subject's legally acceptable representative, and after the subject or the subject's legally acceptable representative has orally consented to the subject's participation in the study and, if capable of doing so, has signed and personally dated the ICF, the witness should sign and personally date the consent form.

The subject will be asked to sign an ICF at Screening Visit before any study-specific procedures are performed. No subject can enter the study before his/her informed consent has been obtained. An unsigned copy of an IRB/IEC-approved ICF will be prepared in accordance with Mount Sinai's PPHS Standard consent template. Each subject must sign an approved ICF before study participation. The form must be signed and dated by the appropriate parties.

The original, signed ICF for each subject will be verified by the investigator, kept on file and stored in a secured cabinet.

### Privacy

Prospective subjects will be initially approached by their attending epileptologist. Research team will only approach prospective participants after the study has been introduced by their treating epileptologist and they indicate interest. Subjects will only be approached during clinical visits in private offices/exam rooms. Any communication will be conducted in a private room, and with the subject and/or caregiver who provided permission or someone they have agreed can participate in the decision making for the subject. Study team will only contact subjects using their preferred phone number. Subjects will be consented in a quiet room to avoid conversation being overheard by a third party.

### REFERENCES

Marije aan het Rot M, Collins K A, Murrough J W, Perez A M, Reich D L, Charney D S, Mathew S J. Safety and efficacy of repeated-dose intravenous ketamine for treatment-resistant depression. Biol Psychiatry. 2010 Jan. 15; 67(2):139-45. doi: 10.1016/j.biopsych.2009.08.038. PMID: 19897179.
Aram J A, Martin D, Tomczyk M, Zeman S, Millar J, Pohler G, Lodge D. Neocortical epileptogenesis in vitro: studies with N-methyl-D-aspartate, phencyclidine, sigma and dextromethorphan receptor ligands. J Pharmacol Exp Ther. 1989 January;248(1):320-8. PMID: 2536430.
Berman R M, Cappiello A, Anand A, Oren D A, Heninger G R, Charney D S, et al. Antidepressant effects of ketamine in depressed patients. Biol Psychiatry [Internet]. 2000; 47:351-4.
Brodie M J. Road to refractory epilepsy: the Glasgow story. Epilepsia. 2013 May;54 Suppl 2:5-8. doi: 10.1111/epi.12175. PMID: 23646962.
Dingledine R, Borges K, Bowie D, Traynelis S F. The glutamate receptor ion channels. Pharmacol Rev. 1999 March;51(1):7-61. PMID: 10049997.
Esaian D, Joset D, Lazarovits C, Dugan P C, Fridman D. Ketamine continuous infusion for refractory status epilepticus in a patient with anticonvulsant hypersensitivity syndrome. Ann Pharmacother 2013; 47:1569-76.
Feder A, Parides M K, Murrough J W, Perez A M, Morgan J E, Saxena S, Kirkwood K, Aan Het Rot M, Lapidus K A, Wan L B, Iosifescu D, Charney D S. Efficacy of intravenous ketamine for treatment of chronic posttraumatic stress disorder: a randomized clinical trial. JAMA Psychiatry. 2014 Jun;71(6):681-8. doi: 10.1001/jamapsychiatry.2014.62. PMID: 24740528.
Freeman F G, Jarvis M F, Duncan P M. Phencyclidine raises kindled seizure thresholds. Pharmacol Biochem Behav. 1982 June;16(6):1009-11. doi: 10.1016/0091-3057(82)90062-4. PMID: 7111344.
Fujikawa D G. Neuroprotective effect of ketamine administered after status epilepticus onset. Epilepsia. 1995 February;36(2):186-95. doi: 10.1111/j.1528-1157.1995.tb00979.x. PMID: 7821277.
Hesdorffer D C, Ishihara L, Mynepalli L, Webb D J, Weil J, Hauser W A. Epilepsy, suicidality, and psychiatric disorders: a bidirectional association. Ann Neurol. 2012 Aug;72(2):184-91. doi: 10.1002/ana.23601. Epub 2012 Aug 7. PMID: 22887468.
Hsieh C Y, Sung P S, Tsai J J, Hwang C W. Terminating prolonged refractory status epilepticus using ketamine. Clin Neuropharmacol 2010; 33:165-7.
Kapur J, Lothman E W. NMDA receptor activation mediates the loss of GABAergic inhibition induced by recurrent seizures. Epilepsy Res. 1990 March;5(2):103-11. doi: 10.1016/0920-1211(90)90025-q. PMID: 2158437.
Kramer A H. Early ketamine to treat refractory status epilepticus. Neurocrit Care 2012; 16:299-305.
Kramer U, Shorer Z, Ben-Zeev B, Lerman-Sagie T, GoldbergStern H, Lahat E. Severe refractory status epilepticus owing to presumed encephalitis. J Child Neurol 2005; 20:184-7.
Kwan P, Arzimanoglou A, Berg A T, et al. Definition of drug resistant epilepsy: consensus proposal by the ad hoc Task Force of the ILAE Commission on Therapeutic Strategies. Epilepsia 2010; 51:1069-77. [Erratum, Epilepsia 2010; 51:1922.]
Gaspard N, Foreman B, Judd L M, Brenton J N, Nathan B R, McCoy B M, Al-Otaibi A, Kilbride R, Fernández I S, Mendoza L, Samuel S, Zakaria A, Kalamangalam G P, Legros B, Szaflarski J P, Loddenkemper T, Hahn C D, Goodkin H P, Claassen J, Hirsch L J, Laroche S M. Intravenous ketamine for the treatment of refractory status epilepticus: a retrospective multicenter study. Epilepsia. 2013 August; 54(8):1498-503. doi: 10.1111/epi.12247. Epub 2013 Jun 12. PMID: 23758557; PMCID: PMC3731413.
Lang E, Mallien A S, Vasilescu A-N, Hefter D, Luoni A, Riva M A, et al. Molecular and cellular dissection of NMDA receptor subtypes as antidepressant targets. Neurosci Biobehav Rev [Internet]. 2017
Lapidus K A, Levitch C F, Perez A M, Brallier J W, Parides M K, Soleimani L, Feder A, Iosifescu D V, Charney D S, Murrough J W. A randomized controlled trial of intranasal ketamine in major depressive disorder. Biol Psychiatry. 2014 Dec. 15; 76(12):970-6. doi: 10.1016/j.biopsych.2014.03.026. Epub 2014 Apr 3. PMID: 24821196; PMCID: PMC4185009.
Lawn N D, Bamlet W R, Radhakrishnan K, O'Brien P C, So E L. Injuries due to seizures in persons with epilepsy: a population based study. Neurology 2004; 63:1565-70.
McCagh J, Fisk J E, Baker G A. Epilepsy, psychosocial and cognitive functioning. Epilepsy Res 2009; 86:1-14.
Lent J K, Arredondo A, Pugh M A, Austin P N. Ketamine and Treatment-Resistant Depression. AANA J. 2019 October;87(5):411-419. PMID: 31612847.
Mazarati A M, Wasterlain C G. N-methyl-D-asparate receptor antagonists abolish the maintenance phase of self-sustaining status epilepticus in rat. Neurosci Lett. 1999 Apr. 23; 265(3):187-90. doi: 10.1016/s0304-3940(99)00238-4. PMID: 10327162.
Mewasingh L D, Sékhara T, Aeby A, Christiaens F J, Dan B. Oral ketamine in paediatric non-convulsive status epilepticus. Seizure. 2003 October;12(7):483-9. doi: 10.1016/s1059-1311(03)00028-1. PMID: 12967577.
Mohanraj R, Norrie J, Stephen L J, Kelly K, Hitiris N, Brodie M J. Mortality in adults with newly diagnosed and chronic epilepsy: a retrospective comparative study. Lancet Neurol 2006; 5:481-7
Murrough J W, Burdick K E, Levitch C F, Perez A M, Brallier J W, Chang L C, Foulkes A, Charney D S, Mathew S J, Iosifescu D V. Neurocognitive effects of ketamine and association with antidepressant response in individuals with treatment-resistant depression: a randomized controlled trial.
Neuropsychopharmacology. 2015 Mar. 13; 40(5):1084-90. doi: 10.1038/npp.2014.298. PMID: 25374095; PMCID: PMC4367458.
Murrough J W, Iosifescu D V, Chang L C, Al Jurdi R K, Green C E, Perez A M, Iqbal S, Pillemer S, Folks A, Shah A, Charney D S, Mathew S J. Antidepressant efficacy of ketamine in treatment-resistant major depression: a two-site randomized controlled trial. Am J Psychiatry. 2013 Oct;170(10):1134-42. doi: 10.1176/appi.ajp.2013.13030392. PMID: 23982301; PMCID: PMC3992936.
Murrough J W, Perez A M, Pillemer S, Stern J, Parides M K, aan het Rot M, Collins K A, Mathew S J, Charney D S, Iosifescu D V. Rapid and longer-term antidepressant effects of repeated ketamine infusions in treatment-resistant major depression. Biol Psychiatry. 2013 Aug. 15; 74(4):250-6. doi: 10.1016/j.biopsych.2012.06.022. Epub 2012 Jul 27. PMID: 22840761; PMCID: PMC3725185.
Niciu M J, Ionescu D F, Richards E M, Zarate C A Jr. Glutamate and its receptors in the pathophysiology and treatment of major depressive disorder. J Neural Transm (Vienna). 2014 Aug;121(8):907-24. doi: 10.1007/s00702-013-1130-x. Epub 2013 Dec 8. PMID: 24318540; PMCID: PMC4048804.
Prüss H, Holtkamp M. Ketamine successfully terminates malignant status epilepticus. Epilepsy Res. 2008 December;82(2-3):219-22. doi: 10.1016/j.eplepsyres.2008.08.005. Epub 2008 Sept. 19. PMID: 18804344.
Rosati A, De Masi S, Guerrini R. Ketamine for Refractory Status Epilepticus: A Systematic Review. CNS Drugs. 2018 November; 32(11):997-1009. doi: 10.1007/s40263-018-0569-6. PMID: 30232735.
Tarocco A, Ballardini E, Garani G. Use of ketamine in a newborn with refractory status epilepticus: a case report. Pediatr Neurol 2014; 51:154-6.
Scott A J, Sharpe L, Hunt C, Gandy M. Anxiety and depressive disorders in people with epilepsy: A meta-analysis. Epilepsia. 2017 June;58(6):973-982. doi: 10.1111/epi.13769. Epub 2017 May 3. PMID: 28470748.
Sheth R D, Gidal B E. Refractory status epilepticus: response to ketamine. Neurology 1998; 51:1765-6.
Synowiec A S, Singh D S, Yenugadhati V, Valeriano J P, Schramke C J, Kelly K M. Ketamine use in the treatment of refractory status epilepticus. Epilepsy Res 2013; 105:183-8.
Ubogu E E, Sagar S M, Lerner A J, Maddux B N, Suarez J I, Werz M A. Ketamine for refractory status epilepticus: a case of possible ketamine-induced neurotoxicity. Epilepsy Behav 2003; 4:70-5.
Walker M C, Howard R S, Smith S J, Miller D H, Shorvon S D, Hirsch S D. Diagnosis and treatment of status epilepticus on a neurological intensive care unit. QJM 1996; 89:913-20.
Yeh P S, Shen H N, Chen T Y. Oral ketamine controlled refractory nonconvulsive status epilepticus in an elderly patient. Seizure 2011; 20:723-6.
Zarate C A Jr, Singh J B, Carlson P J, Brutsche N E, Ameli R, Luckenbaugh D A, Charney D S, Manji H K. A randomized trial of an N-methyl-D-aspartate antagonist in treatment-resistant major depression. Arch Gen Psychiatry. 2006 August; 63(8):856-64. doi: 10.1001/archpsyc.63.8.856. PMID: 16894061.
Zeiler F A, Kaufmann A M, Gillman L M, West M, Silvaggio J. Ketamine for medically refractory status epilepticus after elective aneurysm clipping. Neurocrit Care 2013; 19:119-24.
Zeiler F A, Teitelbaum J, Gillman L M, West M. NMDA antagonists for refractory seizures. Neurocrit Care 2014; 20:502-13.

The disclosures of all publications cited herein are expressly incorporated herein by reference, each in its entirety, to the same extent as if each were incorporated by reference individually.

A number of embodiments of the invention have been described. Nevertheless, it will be understood that various modifications may be made without departing from the spirit and scope of the invention.

1. A method of treating drug resistant epilepsy in a subject in need of treatment thereof, comprising intranasally administering a therapeutically effective amount of ketamine, or a pharmaceutically acceptable salt thereof, to the subject.

2. A method of treating epilepsy in a subject in need of treatment thereof, comprising intranasally administering a therapeutically effective amount of ketamine, or a pharmaceutically acceptable salt thereof, to the subject; wherein the subject experiences substantially no anesthesia after administration of the ketamine or pharmaceutically acceptable salt thereof and was previously administered an anti-seizure drug.

3. (canceled)

4. (canceled)

5. The method of claim 1, wherein substantially no improvement in the duration, severity, and/or frequency of seizures was observed in the subject after administration of the anti-seizure drug.

6. (canceled)

7. The method of claim 5, wherein the clinical record indicates that substantially no improvement in the duration, severity, and/or frequency of seizures was observed in the subject after intra-nasal administration of the anti-seizure drug.

8. (canceled)

9. A method of selecting a subject for treatment including administration of a therapeutically effective amount of ketamine, or a pharmaceutically acceptable salt thereof, the method comprising:
identifying a subject that has epilepsy and was administered an anti-seizure drug;
determining that there was substantially no improvement in the duration, severity, and/or frequency of seizures in the subject after administration of the anti-seizure drug; and
selecting the subject for treatment including intra-nasal administration of a therapeutically effective amount of ketamine, or a pharmaceutically acceptable salt.

10. The method of claim 3, wherein the anti-seizure drug is selected from the group consisting of: topiramate, valproic acid, felbamate, rufinamide, stiripentol, fenfluramine, clobazam, clonazepam, lorazepam, gabapentin, pregabalin, retigabine, phenytoin, carbamazepine, oxcarbazepine, eslicarbazepine acetate, lamotrigine, lacosamide, zonisamide, levetiracetam, brivaracetam, ethosuximide, perampanel, phenobarbital, primidone, epidiolex, cenobamate, and tiagabine.

11. The method of claim 1, wherein after intranasally administering the ketamine the frequency, severity, and/or duration of one or more symptoms of the epilepsy is reduced.

12. The method of claim 10, wherein the one or more symptoms of the epilepsy are selected from the group consisting of: focal seizures, generalized seizures, non-convulsive seizures, involuntary muscle contractions, auras, Jacksonian march, sensory disturbances, lip smacking, object lifting, involuntary vocalizations, erratic breathing, blue skin, loss of bladder or bowel control, tongue biting, or any combination thereof.

13. (canceled)

14. (canceled)

15. The method of claim 12, wherein the generalized seizures are selected from the group consisting of: tonic-clonic, tonic, clonic, myoclonic, absence, and atonic seizures.

16. The method of claim 1, wherein the subject experiences substantially no anesthesia after intra-nasal administration of the ketamine.

17. The method of claim 1, wherein the therapeutically effective amount is a sub-anesthetic dose of ketamine.

18. The method of claim 1, wherein the therapeutically effective amount is from about 10% to about 30% of the dose required to produce anesthesia in a subject.

19. The method of claim 1, wherein the subject receives multiple intra-nasal doses of ketamine at spaced apart intervals.

20. (canceled)

21. The method of claim 1, wherein the ketamine is formulated with a pharmaceutically acceptable carrier or diluent.

22. (canceled)

23. The method of claim 21, wherein the carrier or diluent comprises sterile phosphate buffered saline solution, bacteriostatic water, aqueous glycine, or any combination thereof.

24. The method of claim 21, wherein the therapeutically effective amount is between about 0.1 and about 2.0 mg/kg.

25. The method of claim 20, wherein the therapeutically effective amount is about 0.5 ml/kg.

26. The method of claim 1, comprising intranasally administering the ketamine once per week.

27. The method of claim 1, comprising intranasally administering the ketamine three times per week.

28. (canceled)

29. (canceled)

30. The method of claim 1, wherein the ketamine is administered as a spray

31. The method of claim 30, wherein the ketamine is formulated as a solution or suspension.

32. (canceled)

33. The method of claim 30, wherein the ketamine is formulated as a dry powder.

34. The method of claim 30, wherein the ketamine is contacted with the nasal mucosa.

35. The method of claim 30, wherein the ketamine is intranasally administered by means of a device comprising a metered dose inhaler.

36. The method of claim 30 wherein the ketamine or pharmaceutically acceptable salt thereof is administered by means of a device comprising a nasal spray inhaler containing an aerosol spray formulation of ketamine and a pharmaceutically acceptable dispersant, wherein the device is metered to disperse an amount of the aerosol formulation by forming a spray that contains a dose of ketamine effective to treat epilepsy but which dose of ketamine is determined by a physician or medical care provider to be below a dose that causes anesthesia.

37. The method of claim 30, wherein the ketamine is formulated with a pharmaceutically acceptable carrier or diluent.

38. (canceled)

39. The method of claim 30, wherein the ketamine is formulated with a mucosal penetration enhancer.

40. (canceled)

41. The method of claim 30, wherein the therapeutically effective amount is between about 0.05 and about 0.7 mg/kg.

42. The method of claim 30 wherein the therapeutically effective amount is about 0.5 ml/kg.

43. The method of claim 1, wherein the ketamine is administered 3 times per week.

44. (canceled)

45. The method of claim 1 wherein the ketamine is administered during a seizure, and the subject transitions to a postictal state or a normal state within about 10 minutes after administration of the ketamine.

46. (canceled)

47. The method of claim 1, wherein the ketamine is esketamine.

48. The method of claim 1 wherein after intranasally administering the ketamine the frequency of seizures is reduced.

49. (canceled)

50. The method of claim 1, wherein the reduction in seizure frequency is at least about 10%.

51. (canceled)

52. (canceled)

53. The method of claim 1, wherein the subject was identified or diagnosed as having depression and/or anxiety before administering the ketamine.

54. The method of claim 1, wherein the Neurological Disorders Depression Inventory for Epilepsy (NDDI-E) score of the subject is lower after intranasally administering the ketamine.

55. The method of claim 54, wherein the NDDI-E score is at least 1 point lower after administering the ketamine.

56. (canceled)

57. The method of claim 1, wherein the Generalized Anxiety Disorder 7 (GAD-7) score of the subject is lower after intranasally administering the ketamine.

58. The method of claim 57, wherein the GAD-7 score is at least 10% lower after administering the ketamine.

59. (canceled)

60. The method of claim 1, wherein the Anxiety, Depression and Mood Scale (ADAMS) score of the subject is lower after administering the ketamine.

61. The method of claim 60, wherein the ADAMS score is at least 10% lower after administering the ketamine.

62. (canceled)

63. The method of claim 1, wherein the Quality of Life in Epilepsy Inventory-10 (QOLIE-10) score of the subject is lower after administering the ketamine.

64. (canceled)

65. The method of claim 63, wherein the QOLIE-10 score is at least 30% lower after administering the ketamine.

66. The method of claim 1, comprising administering a second therapeutic agent.

67. The method of claim 66, wherein the second therapeutic agent is an anti-seizure drug.
